(19) United States
(12) Patent Application Publication
(10) Pub. No.: US 2013/0085413 A1
(43) Pub. Date: Apr. 4, 2013

(54) ANATOMICAL-POSITIONING APPARATUS AND METHOD WITH AN EXPANDABLE DEVICE

(71) Applicants: Oded Tsamir, Tel Aviv (IL); Lior Margalit, Tel Aviv (IL)

(72) Inventors: Oded Tsamir, Tel Aviv (IL); Lior Margalit, Tel Aviv (IL)

(21) Appl. No.: 13/703,427

(22) PCT Filed: Jun. 13, 2011
(86) PCT No.: PCT/IL11/00465

§ 371 Date: Dec. 11, 2012

Related U.S. Application Data
(60) Provisional application No. 61/354,225, filed on Jun. 13, 2010.

(51) Int. Cl. A61B 5/107; A61M 19/00; A61M 25/01; A61B 10/02; A61M 5/00

(52) U.S. Cl. CPC A61B 5/1075; A61B 10/0233; A61M 5/00; A61M 25/01; A61M 19/00
USPC 600/567; 600/587; 604/506

(57) ABSTRACT

An anatomical-positioning apparatus for acquiring mechanical data from a tissue to facilitate determining type of the tissue and transition between different tissues and cavities. The anatomical-positioning apparatus includes a cannula, having a tip an expandable device having a contracted form size and an expanded form size, wherein the expanded form size is substantially larger than the contracted form size. The anatomical-positioning apparatus further includes an introducer, having a longitudinal axis and a distal end, facilitating the introduction of the expandable device into the tissue, the expandable device being in a contracted state, an expanding-mechanism for expanding and contracting the expandable device, and a sensor for measuring physical parameters associated with the expandable device. An aspect of the present invention is to provide a method for acquiring mechanical data from a tissue by an expandable device, while the expandable device exerts pressure onto portions of the tissue.

### CROSS REFERENCE TO RELATED APPLICATIONS

This application claims the benefit under 35 USC 119(e) from U.S. provisional application 61/354,225, filed on Jun. 13, 2010, the disclosure of which is incorporated by reference for all purposes as if fully set forth herein.

### FIELD OF THE INVENTION

This invention relates to invasive medical devices and more particularly, the present invention relates to anatomical-positioning medical devices for controlled advancement thorough multiple tissues of various types and cavities disposed there between, until reaching a target position. For example, an Epidural anesthesia device to provide controlled access to the Epidural space, while preventing puncturing the Dura mater.

### BACKGROUND OF THE INVENTION

In medical procedures, needle insertion and localization are issues of great importance. Misplacement of a needle tip or a trocar can harm essential tissues such as blood vessels, nerves or internal organs and lead to major complications. For example, during a procedure of Epidural anesthesia the needle tip should be placed inside the Epidural space (ES), to thereby facilitate administration of anesthetic agents. This has become a common and effective procedure for controlling pain during childbirth, major surgery, and chronic back pain.

Reference is made to FIG. 1, an illustration of the final stage of an Epidural anesthesia procedure. The tip of a needle 92 of a device 90, for administering a medication into Epidural space 70, is disposed inside Epidural space 70, after being inserted through skin 30 and advanced between Spinous process 55 and through the subcutaneous fat layer 40, the Supraspinous Ligament 50, the Interspinous Ligament 52 and the Ligamentum Flavum (LF) 60.

Overshooting of the tip of the needle beyond the ES may puncture the Dura mater 80 causing a leak of the cerebral-spinal fluid (CSF) from spinal cord 85 into the ES, leading to severe headaches (post dural puncture headaches syndrome).

The majority of current injection techniques are “blind” techniques, mainly tactile based. For example, the main technique of Epidural block is based on the “loss of resistance technique” (LORT). In LORT, a fluid or air filled syringe is attached to a needle. While the needle is advanced through different layers in the insertion site, the physician taps on the syringe. Inside dense Ligament layers, the physician fills a strong resistance, but when crossing the LF 60 and entering the ES 70, there is a substantial loss of resistance so that the fluid or air from the syringe can be easily pushed into the low-pressured ES 70, thus signaling the physician to stop advancing needle 92.

In practice, the LORT holds some major disadvantages. Because of the elastic properties of the LF 60, the elastic fibers are pushed by the needle and are stretched into the ES 70. For this reason, the rupture of these fibers takes place deep inside the ES 70 increasing the risk of an overshooting of the needle tip into the Dura mater 80. Moreover, the resolution of the non-controlled advancement-increments of the needle-tip is very limited and differs extensively from one physician to another. Another disadvantage of LORT is the relatively high risk of a false loss of resistance, taking place for instance inside the LF 60 due to a small space between adjacent fibers.

FIGS. 2a-2d illustrate cross sectional views of the stages of a typical Epidural anesthesia procedure, including the penetration of Ligamentum Flavum 60 and including entering into Epidural space 70. When needle 95 is advanced through Ligamentum

Flavum 60, the elastic fibers of Ligamentum Flavum 60 are stretched by pushing pressure exerted by needle 95 deep into Epidural space 70, before entering Epidural space 70 (see FIGS. 2b and 2c). When the fibers reach a certain displacement, Ligamentum Flavum 60 ruptures and the needle penetrates into Epidural space 70, as depicted in FIG. 2d, typically stopping a short distance (d₁) from Dura mater 80. The displacement required for the fiber to rupture differs from one person to another due to physiologic variations in Ligamentum Flavum elasticity, thickness and other factors. However, using the prior art technique has an extensive risk of accidently puncturing the Dura mater due to overshooting of the needle.

U.S. Pat. No. 5,188,594, given to Michael Zilberstein, provides a device and method for administering medicine into the Epidural space by first supplying air through a valve connected to a needle and provided with an inflatable element so that before the needle reaches the Epidural space the inflatable element is inflated. The needle is advanced and when the needle reaches the Epidural space 70 the inflatable element is deflated, which deflation is, typically, observed by the physician (or his/her stuff); medication is then administered through the valve and through the needle into the Epidural space.

There is a need for and it would be advantageous to have an injection device that can overcome the disadvantages mentioned hereabove, including the disadvantages of the LORT, and is relatively a low cost solution.

The terms “Epidural anesthesia procedure” and “Epidural block procedure” are used herein interchangeably.

### SUMMARY OF THE INVENTION

By way of introduction, the principal intentions of the present invention include providing an anatomical-positioning apparatus, such as an injection device, that facilitates acquiring mechanical data from a mammalian tissue by an expandable device, while the expandable device exerts pressure onto portions of said mammalian tissue. Thereby facilitating to determine the type of a mammalian tissue, as well as determining transition between different mammalian tissues and cavities. An aspect of the present invention is to provide safe methods for advancing an introducer, typically a sharp introducer, from an elastic tissue to a soft tissue, such as from the Ligamentum Flavum to the Epidural space.

According to the teachings of the present invention there is provided an anatomical-positioning apparatus for acquiring mechanical data from a mammalian tissue to facilitate determining type of the tissue and transition between different tissues and cavities. The anatomical-positioning apparatus includes a cannula, having a tip at the distal section of the cannula, an expandable device having a contracted form size, when in a contracted state, and an expanded form size, when in an expanded state, wherein the expanded form size is substantially larger than the contracted form size. The anatomical-positioning apparatus further includes an introducer, having a longitudinal axis and a distal end, facilitating the introduction of the expandable device into the mammalian tissue, the expandable device being in the contracted state, an expanding-mechanism for expanding and contracting the expandable device, and a sensor for measuring physical parameters associated with the expandable device, to thereby provide sensed data.

The sensed data is acquired while the expandable device exerts pressure onto portions of the mammalian tissue, to thereby facilitating determining the type of tissue that is in contact with the expandable device and to determine a transition between different tissues and cavities. The sensor includes one or more sensing devices selected from the group of devices consisting of a pressure sensor, strain gauge, force sensor, tactile sensor, displacement sensor, volume sensor, flow sensor and a piezoelectric transducer. Optionally, the sensing device includes an array of sensors disposed on the expandable device.

Typically, the sensed data is indicated by one or more indication devices selected from the group of devices consisting of a display, a gage, a light indicator, an audio indicator and a tactile indicator.

Optionally, the sensed data includes a measurement of the advancement of the cannula against the mammalian tissue, and wherein the displacement of the expandable device, along the longitudinal axis, is inferred from the sensed data.

Optionally, the sensed data includes a one-dimensional measurement of the displacement of the expandable device, during the expanding against the mammalian tissue or contracting away from the mammalian tissue.

Optionally, the sensed data includes one or more measurements selected from a group of measurements consisting of the pressure inside the expandable device, the volume of gas or fluid inside the expandable device, an external force applied on the expandable device by the mammalian tissue and the spatial pressure or spatial force applied on the expandable device by the mammalian tissue, while the expandable device exerts pressure onto portions of the mammalian tissue.

Optionally, the sensed data facilitates determining one or more measurements selected from a group of measurements consisting of the volume-pressure work performed by the expandable device, a volume-pressure profile of the expandable device, the work being done by the expandable device and the force-displacement profile of the expandable device, while the expandable device exerts pressure onto portions of the mammalian tissue.

The expandable device is securely attached to the tip of the cannula or on the external circumference of the cannula, proximal to the tip of the cannula and when the expandable device expands from the contracted state to the expanded form size, the expandable device is disposed outside the introducer, and a blunt contact surface is formed at the external surface of the expanded expandable device.

The expandable device protects the mammalian tissue or an anatomical structure ahead of the mammalian tissue, from being punctured by the introducer of the expandable device. The cannula protects the expandable device from being punctured by the introducer. Typically, the expandable device expands and contracts under fluid or gas pressure provided by the expanding-mechanism through the cannula.

In variations of the present invention, the introducer is an openable introducer, having an openable sharp tip, wherein the openable sharp tip of the openable introducer has a closed state and an open state, and wherein the expandable device is trapped inside the openable sharp tip, the openable sharp tip being in a closed state. Preferably, the expandable device facilitates the openable sharp tip to remain in the closed state, when the expandable device is loaded by a preconfigured expanding force and the sharp tip is situated in a high density tissue. The expandable device facilitates the opening of the openable sharp tip, when the expandable device is loaded be a preconfigured expanding force and the openable sharp tip enters a low density tissue or cavity. The closed state facilitates advancement of the openable introducer through the mammalian tissue, while the open state prevents advancement of the openable introducer through the mammalian tissue. It should be noted that the expandable device also protects soft tissues ahead of expandable device.

Optionally, the introducer is selected from the group consisting of a veress needle, an epidural needle, a biopsy needle, a trocar, a cannula, a catheter, a Tuohy type needle and a surgical instrument.

Optionally, the expandable device is selected from a group of devices consisting of a balloon, a membrane, a diaphragm, a spring and a flexible device. Optionally, the expandable device includes one or more mechanical devices selected from a group of devices consisting of a spring and a balloon enclosed in a conical element, wherein the conical element protects the balloon from being punctured.

Optionally, the cannula and the expandable device is introduced on-demand and can be withdrawn during a procedure in order to enable utilization of other tools inside the mammalian tissue.

Optionally, the cannula includes a double lumen, wherein a first lumen facilitates a pathway for the fluid or gas into the expandable device, and wherein a second lumen facilitates a pathway for one or more devices selected from a group of devices consisting of a thin needle, a catheter, an optical fiber, an electrode and a surgical instrument.

Optionally, the cannula includes a single lumen facilitating a pathway for the fluid or gas into the expandable device, and wherein the single lumen facilitating a pathway for an optical fiber or a miniature camera, to thereby facilitate visualization of the mammalian tissue through the expandable device, the expandable device being in contact with the mammalian tissue.

Preferably, the anatomical-positioning apparatus further includes an advancement mechanism for incrementally advancing, in a controlled manner, one or more devices selected from the group consisting of the introducer, the cannula and the expandable device. Optionally, the advancement mechanism is selected from the group of devices consisting of a motor based device, hydraulic device, gear based device and a screw based device. Optionally, the introducer can be advanced incrementally together with the cannula and the expandable device. Optionally, the introducer can be advanced incrementally with respect to the cannula and the expandable device.

Preferably, the anatomical-positioning apparatus further includes a processor facilitated to record and analyze the sensed data, to thereby determine the type of tissue that is in contact with the expandable device and to determine the transition between tissues and cavities, wherein the sensed data is acquired while the expandable device is expanding, contracting or in a steady expansion level.

It is an aspect of the present invention to provide a method for acquiring mechanical data from a mammalian tissue by an expandable device, while the expandable device exerts pressure onto portions of the mammalian tissue. The method includes the steps of:
a) providing an anatomical-positioning apparatus including a cannula, having a tip at the distal section of the cannula; an expandable device having a contracted form size, when in a contracted state, and an expanded form size, when in an expanded state, wherein the expanded form size is substantially larger than the contracted form size, and wherein the expandable device is securely attached to the tip of the cannula or on the external circumference of the cannula, proximal to the tip of the cannula; an introducer, having a distal end, facilitating the introduction of the expandable device into the mammalian tissue, the expandable device being in the contracted state; an expanding-mechanism for expanding and contracting the expandable device; and a measuring-mechanism for measuring physical parameters associated with the expandable device, while the expandable device exerts pressure onto portions of the mammalian tissue
b) inserting the introducer inside the mammalian tissue, wherein the distal end of the introducer enters first;
c) advancing the expandable device through the introducer, using the cannula;
d) expanding the expandable device outside the distal end of the introducer;
e) measuring the physical parameters associated with the expandable device, while the expandable device exerts pressure onto portions of the mammalian tissue, thereby creating sensed data.

Preferably, the method further includes the step of analyzing the sensed data, using a processor, thereby determining the type of the mammalian tissue being in contact with the expandable device, and determining a transition between different mammalian tissues and cavities.

Optionally, in the method, the sensed data includes one or more measurements selected from a group of measurements consisting of the displacement of the expandable device and cannula, the pressure inside the expandable device, the volume of gas or fluid inside the expandable device, the external force applied on the expandable device by the mammalian tissue and the spatial pressure or spatial force applied on the expandable device by the mammalian tissue, while the expandable device exerts pressure onto portions of the mammalian tissue.

Optionally, in the method, the sensed data facilitates determining one or more measurements selected from a group of measurements consisting of the volume-pressure work performed by the expandable device, the volume-pressure profile of the expandable device, the work being done by the expandable device and the force-displacement profile of the expandable device, while the expandable device exerts pressure onto portions of the mammalian tissue.

Optionally, in the method, the measuring of the sensed data is performed during states selected from a group of states consisting of an expansion of the expandable device, a contraction of the expandable device and a displacement of the cannula and the expandable device, while the expandable device is expanded.

It is another aspect of the present invention to provide a spring-back method for a controlled insertion of an introducer into and through an elastic mammalian tissue. The spring-back method includes the steps of:
a) providing a device including a cannula, having a tip at the distal section of the cannula; an expandable device having a contracted form size, when in a contracted state, and an expanded form size, when in an expanded state, wherein when in expanded state, the expandable device has a front end; wherein the expanded form size is substantially larger than the contracted form size; and wherein the expandable device is securely attached to the tip of the cannula or on the external circumference of the cannula, proximal to the tip of the cannula; an introducer, having a sharp tip at the distal end, facilitating the introduction of the expandable device into the mammalian tissue, the expandable device being in the contracted state; an expanding-mechanism for expanding and contracting the expandable device; and an advancement mechanism for incrementally advancing, in a controlled manner, devices selected from the group consisting of the introducer, the cannula and the expandable device;
b) expanding the expandable device by the expanding-mechanism, outside the tip of the introducer, whereby the expandable device exerts pressure onto portions of the mammalian tissue, wherein the mammalian tissue is initially in an undistorted position;
c) advancing the introducer incrementally to a preconfigured position situated beyond the undistorted position of the mammalian tissue, the mammalian tissue being pushed by the expandable device, wherein the tip of the introducer is posterior to the instantaneous position of the front end of the expandable device, with respect to the elastic tissue;
d) holding the introducer in secured position; and
e) contracting the expandable device at a preconfigured rate, thereby facilitating the mammalian tissue, being elastic, to spring back powerfully towards the undistorted position of the mammalian tissue, thereby penetrating the mammalian tissue by the tip of the introducer, being disposed beyond the undistorted position of the mammalian tissue.

Optionally, in the spring-back method, the incremental advancing of the introducer is performed while also incrementally advancing the expandable device, wherein the expandable device is expanded.

Optionally, in the spring-back method, the incremental advancing of the introducer is performed, while the expandable device is expanded and in steady position, wherein the introducer is incrementally displaced beyond the undistorted position of the mammalian tissue, and wherein the introducer tip is kept at a shorter distance from the mammalian tissue, with respect to steady position of the front end of the expandable device.

It is another aspect of the present invention to provide a method for a controlled insertion of a cannula, using an inner needle, into and through an elastic mammalian tissue, the method including the steps of:
a) providing a device including a cannula, having a double lumen and a tip at the distal section of the cannula; an expandable device having a contracted form size, when in a contracted state, and an expanded form size, when in an expanded state, wherein when in expanded state, the expandable device has a front end; wherein the expanded form size is substantially larger than the contracted form size; and wherein the expandable device is securely attached on the external circumference of the cannula, proximal to the tip of the cannula; an introducer, facilitating the introduction of the expandable device into the mammalian tissue, the expandable device being in the contracted state; an expanding-mechanism for expanding and contracting the expandable device; an inner needle being introduced through the cannula; and an advancement mechanism for incrementally advancing, in a controlled manner, devices selected from the group consisting of the introducer, the cannula, the inner needle and the expandable device;
b) Advancing the cannula together with the inner needle outside distal tip of the introducer, wherein the tip of the inner needle is secured inside the cannula near the distal end of the cannula;
c) expanding the expandable device by the expanding-mechanism, whereby the expandable device exerts pressure onto portions of the mammalian tissue, wherein the mammalian tissue is initially in an undistorted position;
d) advancing the cannula together with the inner needle incrementally to a preconfigured position wherein the distal end of the cannula together with the tip of the inner needle are situated beyond the undistorted position of the mammalian tissue, the mammalian tissue being pushed by the expandable device;
e) locking the inner needle and the cannula in a secured position;
f) contracting the expandable device at a preconfigured rate, while unlocking the cannula, thereby facilitating the mammalian tissue, being elastic, to spring back powerfully towards the undistorted position of the mammalian tissue, while pushing backwards the cannula, thereby penetrating the mammalian tissue by the tip of the inner needle, being disposed beyond the undistorted position of the mammalian tissue;
g) incrementally advancing the cannula over the inner needle to a position wherein the distal end of the cannula shields the tip of the inner needle.

Optionally, the insertion method further includes the step of expanding the expandable device by the expanding-mechanism, thereby anchoring the cannula in position, upon transition of the expandable device from the elastic tissue into a different tissue or cavity.

It is yet another aspect of the present invention to provide a method for a controlled insertion of an openable introducer into and through a mammalian tissue, the method including the steps of:
a) providing a device including a cannula, having a double lumen; an openable introducer, including an openable sharp tip having a closed state and an opened state, wherein the closed state facilitates advancement of the openable introducer through the mammalian tissue; and wherein the open state prevents advancement of the openable introducer through the mammalian tissue; an expandable device having a contracted form size, when in a contracted state, and an expanded form size, when in an expanded state.
When in expanded state, the expandable device has a front end. The expanded form size is substantially larger than the contracted form size. The expandable device is securely attached on the external circumference of the cannula, proximal to the tip of the cannula.
The openable introducer facilitates the introduction of the expandable device into the mammalian tissue, the expandable device being in the contracted state.
The expandable device facilitates opening of the tip of the openable introducer when the expandable device is in the expanded state wherein the tip enters a cavity or a low dense tissue;
The device further includes an expanding-mechanism for expanding and contracting the expandable device;
b) advancing the cannula together with the openable introducer into and inside the mammalian tissue, the openable sharp tip being in the closed state;
c) maintaining a constant preconfigured pressure inside the expandable device;
d) advancing the cannula together with the openable introducer until the tip of the openable introducer enters into a low density tissue or cavity; thereby facilitating rapid expanding of the expandable device by the expanding mechanism, the constant preconfigured pressure inside the expandable device being larger than the external pressure applied on the tip of the openable introducer by the low density tissue or cavity.

Optionally, in the method with the openable introducer, the device further includes an advancement mechanism for incrementally advancing, in a controlled manner, devices selected from the group consisting of the openable introducer, the cannula and the expandable device.

### BRIEF DESCRIPTION OF THE DRAWINGS

The present invention will become fully understood from the detailed description given herein below and the accompanying drawings, which are given by way of illustration and example only and thus not limitative of the present invention, and wherein:

FIG. 1 illustrates the Epidural space and surrounding anatomical structures with a needle properly inserted into the Epidural space;

FIGS. 2a-2d (prior art) illustrate cross sectional views of the stages of a typical Epidural anesthesia procedure, including penetration of the Ligamentum Flavum and including entering into the Epidural space;

FIG. 3 is a schematic illustration of an anatomical-positioning apparatus, according to embodiments of the present invention.

FIGS. 4a-4e illustrate cross sectional views of the distal portion of an anatomical-positioning apparatus as shown in FIG. 3, showing the stages of an Epidural anesthesia procedure, including expanding an expandable device inside the Ligamentum Flavum and inside Epidural space;

FIG. 5 illustrates another embodiment of an anatomical-positioning apparatus, according to embodiments of the present invention, having a spring and a conical element, shown in a closed state;

FIG. 6 illustrates the anatomical-positioning apparatus shown in FIG. 5, the conical element being in an opened state;

FIG. 7 is a graph that describes the resistance forces being measured when advancing a plastic tube with a surface area of ˜4 mm² against Ligamentum and against Fat.

FIG. 8 is a graph that describes the resistance forces being measured when advancing a “Tuohy” 18 G needle against Ligamentum and against Fat.

FIG. 9 is a schematic flow chart showing an exemplary method of determining a tissue type, as well as transition between different tissues and cavities, using an anatomical-positioning apparatus, according to embodiments of the present invention.

FIGS. 10a-10e illustrate cross sectional views of the distal portion of an anatomical-positioning apparatus, as shown in FIGS. 5 and 6, showing the stages of a spring-back method of safe penetration of the Ligamentum Flavum and entering into the Epidural space, during an Epidural anesthesia procedure.

FIG. 11 is a schematic flow chart showing an exemplary spring-back method as illustrated in FIGS. 10a-10e.

FIGS. 12a-12d illustrate side views of the distal portion of an anatomical-positioning apparatus, according to variations of the present invention, having a thin-needle being introduced through a cannula for facilitating a spring-back method.

FIGS. 13a-13g illustrate cross sectional views of the distal portion of an anatomical-positioning apparatus, as shown in FIGS. 12a-12d, showing the stages of a spring-back method of safe penetration of the Ligamentum Flavum and entering into the Epidural space, during an Epidural anesthesia procedure.

FIG. 14 is a schematic flow chart showing an exemplary spring-back method as illustrated in FIGS. 13a-13g.

FIG. 15 illustrates the distal section of an anatomical-positioning apparatus, according to variations of the present invention, wherein the introducer includes an openable tip and wherein the openable tip is shown in a closed state.

FIG. 16a illustrates a cross sectional view of the distal portion of the anatomical-positioning apparatus shown in FIG. 15, wherein the introducer penetrates an mammalian tissue, such as the Ligamentum Flavum, and wherein the openable tip is shown in a closed state.

FIG. 16b illustrates a cross sectional view of the distal portion of the anatomical-positioning apparatus shown in FIG. 15, wherein the introducer penetrates a mammalian tissue, such as the Ligamentum Flavum, and wherein the openable tip is shown in an opened state.

FIG. 17 is a schematic flow chart showing an exemplary method safely penetrating the Ligamentum Flavum and entering Epidural space using the anatomical positioning apparatus illustrated in FIGS. 15-16b.

FIG. 18a-18b illustrate side views of an anatomical-positioning apparatus, according to variations of the present invention, having a guide-wire attached to the expandable device for measuring the displacement of the expandable device.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The methods and examples provided herein are illustrative only and not intended to be limiting.

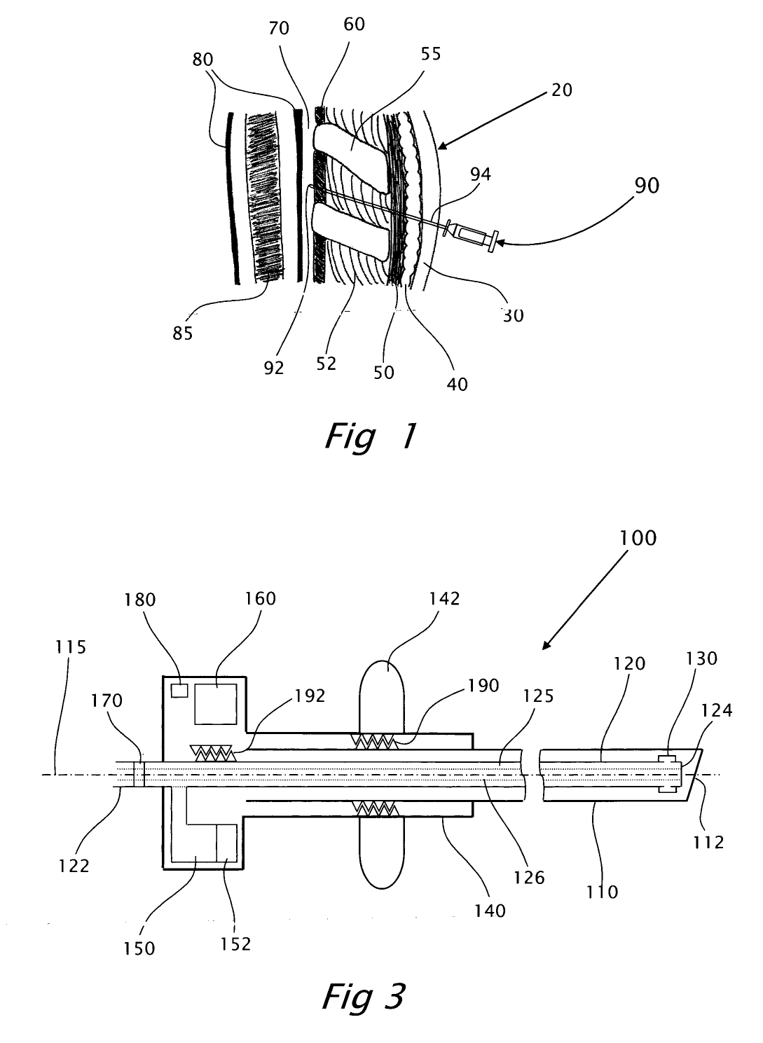

Referring back to FIG. 1 illustrating Epidural space 70 and surrounding anatomical structures with a tip 92 of needle 90 properly inserted into Epidural space 70.

Referring is also to FIG. 3, showing a schematic illustration of an exemplary anatomical-positioning apparatus 100, according to preferred embodiments of the present invention. Anatomical-positioning apparatus 100 includes a housing 140 that secures both a sheath-shaped introducer 110, having a longitudinal axis 115, and an expandable device 130, which expandable device 130 is securely attached to a cannula 120. Anatomical-positioning apparatus 100 further includes a cannula 120, having an opened proximal end 122, a distal end 124, and two lumens, wherein a first lumen 125 facilitates the insertion and evacuation of gas or fluid into expandable device 130, and a second lumen 126 facilitates the insertion of various elongated devices such as a catheter. Expandable device 130 is securely attached to the external circumference of cannula 120, proximal to tip 124 of cannula 120.

In variations of the present invention, cannula 120 has an elongated hollow body (single lumen) facilitating the insertion and evacuation of gas or fluid into expandable device 130, wherein expandable device 130 is securely attached to distal end 124 (tip) of cannula 120. The elongated hollow body of cannula 120 can also provide a pathway for an optical fiber or a miniature camera, to thereby facilitate visualization of a mammalian tissue or cavity through expandable device 130, when expandable device 130 is expanded.

In embodiments, introducer 110 can be a Veress needle, an Epidural needle, a biopsy needle, a trocar, a cannula, a catheter, a Tuohy type needle, a surgical instrument or any other sharp object facilitated to be inserted into at least one mammalian tissue.

It should be noted that the present invention will be described mostly in terms of an anatomical-positioning apparatus for performing a procedure of Epidural anesthesia, including determining the type of one or all tissues the introducer is advancing through, including Interspinous Ligament 52, Ligamentum Flavum 60 and inside Epidural space 70, as well as indicating transition between tissues and cavities. But the present invention is not limited to anatomical-positioning apparatus for performing a procedure of Epidural anesthesia, including determining the type of one or all tissues the introducer is advancing through and indicating transition between tissues and cavities. The present invention includes all anatomical-positioning apparatuses for determining the type of one or more tissues, the introducer is advancing through, as well as indicating transition between different tissues and cavities.

Distal end 112 of introducer 110 is typically, with no limitation, a sharp end. Cannula 120 can be made of any biocompatible material, preferably hard plastic or stainless still. Expandable device 130 can be expanded to a preconfigured volume or shape and have a preconfigured displacement along longitudinal axis 115 of introducer 110 using air or fluid passing through first lumen 125 of cannula 120 and into expandable device 130.

Alternatively, the expandable device can be made of a shape memory alloy, such as NiTi alloy, so that under certain conditions (for instance, heat) it expands. Optionally, the expandable device can be expanded using a mechanical mechanism that is based on springs or any other mechanical element. In one preferred embodiment of the present invention, without limiting the scope of this invention, expandable device 130 is an inflatable balloon that can be made of any elastic material known in the art, such as polyurethane, flexible PVC, PET, nylon, nylon elastomers and thermoplastic elastomers.

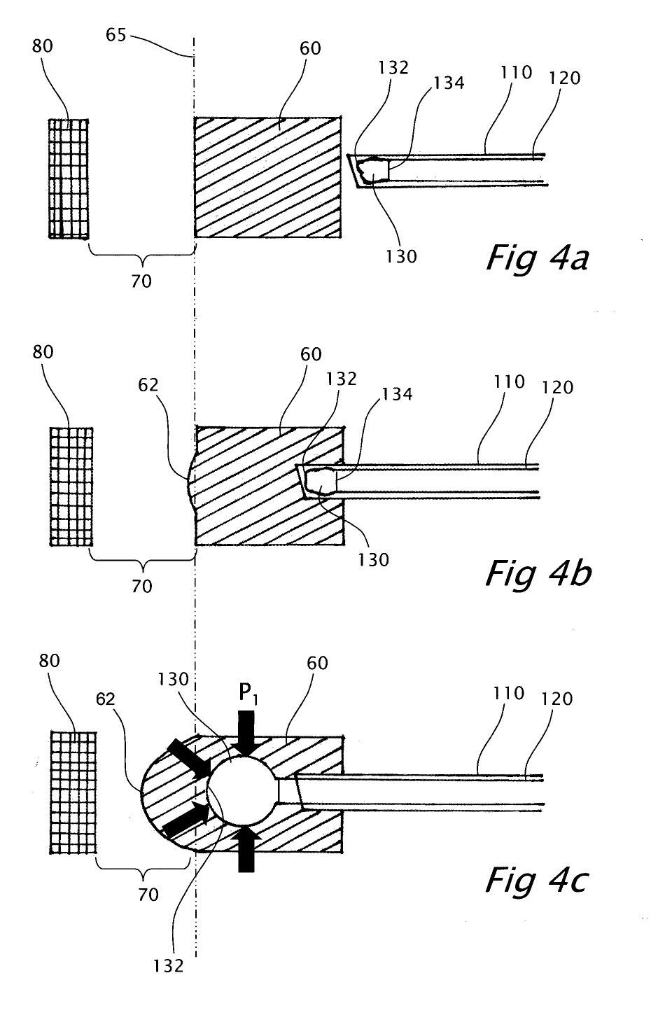

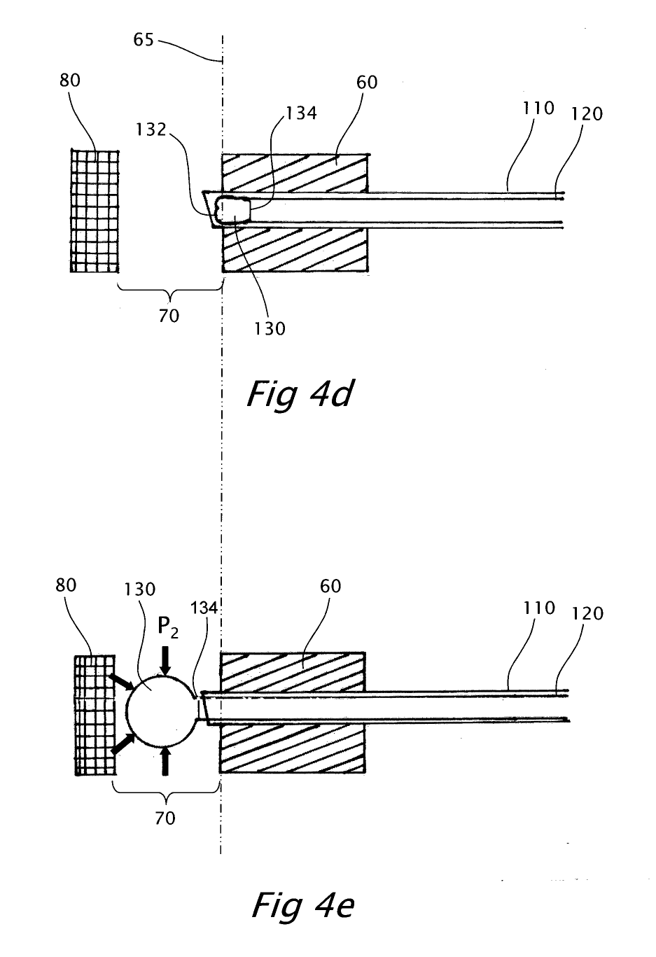

Reference is now made to FIGS. 4a-4e, illustrating cross sectional views of the distal portion of anatomical-positioning apparatus 100, showing the stages of an Epidural anesthesia procedure, including the penetration of Ligamentum Flavum 60 and including entering into Epidural space 70. FIG. 4a illustrates expandable device 130 as a balloon, being in a contracted state, while FIG. 4c illustrates expandable device 130 as a balloon, being in an expanded state. Proximal end 134 of expandable device 130 is securely attached to distal end 124 (tip) of cannula 120.

In the contracted state, balloon 130 is completely deflated, and is protectively disposed inside introducer 110. Balloon 130 is kept deflated by mechanical biasing force or by maintaining partial vacuum-pressure inside balloon 130.

In the expanded state or in a partially expanded state, balloon 130 is popped out of introducer 110, such that introducer 110 does not damage balloon 130. Balloon 130 is kept inflated by providing controlled gas or fluid pressure into balloon 130. When balloon 130 is inflated, distal end 132 of expandable device 130 forms a blunt surface.

Referring back to FIG. 3, Anatomical-positioning apparatus 100 further includes an expanding-mechanism 150 for expanding and contracting expandable device 130. When expandable device 130 is a balloon, the expanding-mechanism, typically, includes a pump (not shown) for controllably pump gas (air) or fluid into and out of balloon 130. Expanding-mechanism 150 may include a container 152 for holding gas or fluid.

Anatomical-positioning apparatus 100 further includes a sensor 170 for measuring physical parameters associated with expandable device 130, while expandable device 130 exerts pressure onto portions of the mammalian tissue being operationally pressed by expandable device 130. The measuring of the physical parameters, associated with expandable device 130, facilitates determining the type of tissue that is in contact with a blunt external surface, typically distal surface 132 of balloon 130, as well as determining transition between tissues and cavities.

The measuring of the physical parameters, associated with expandable device 130, may take place when expandable device 130 expands, when expandable device 130 contracts, or when expandable device 130 is already expanded and in a steady state.

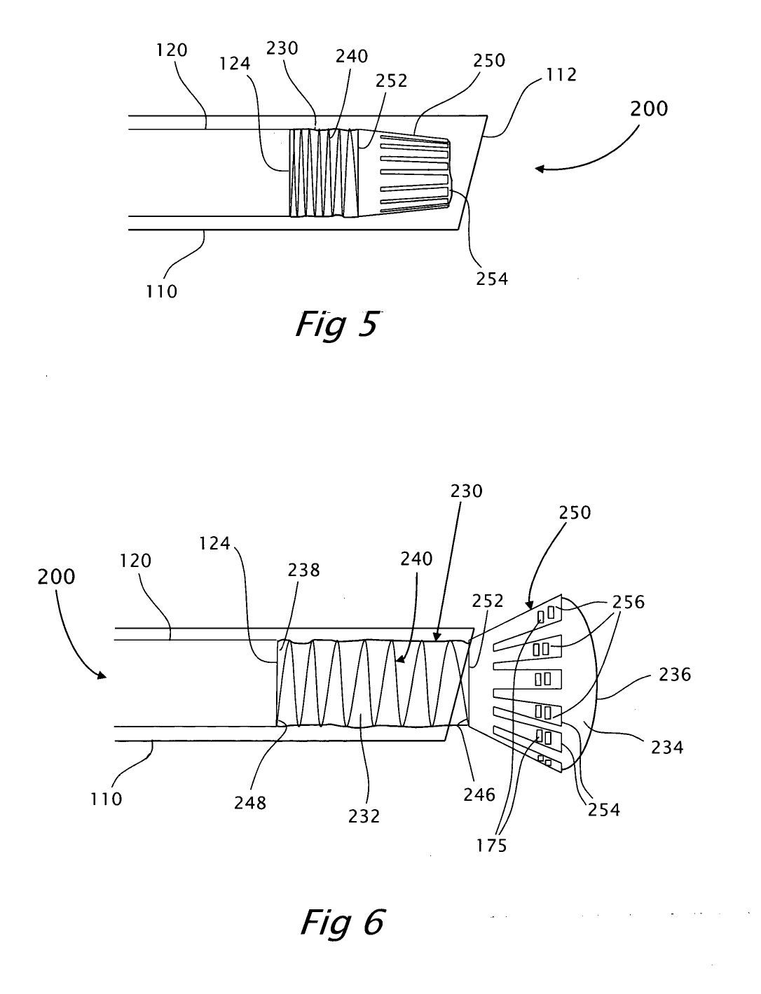

Reference is now made to FIGS. 5-6, illustrating anatomical-positioning apparatus 200 including another embodiment of the expandable device, according to variations of the present invention.

As shown in FIGS. 5 and 6, expandable device 230, such as balloon 230, includes having a proximal section 232 encapsulated inside a biasing element such as a helical spring 240 and a conical element 250 that are securely interconnected. When balloon 230 is inflated to a preconfigured volume, as depicted in FIG. 6, proximal section 232 of balloon 230, having a diameter smaller than the internal diameter of introducer 110, and distal section 234, having a diameter that is typically, with no limitation, 1.1-4 times larger than the internal diameter of introducer 110, and approximately 1.5-2 times larger, in the best mode.

Proximal end 238 of proximal section 232 of balloon 230 is securely attached to proximal end 248 of spring 240, both of which are securely attached to distal end 124 (tip) of cannula 120. The proximal end of distal end section 234 of balloon 230 is disposed at proximal end 252 of conical element 250, wherein conical element 250 has a proximal end 252 and a distal end 254, and wherein proximal end 252 of conical element 250 is securely attached to distal end 246 of spring 240. Proximal end 252 of conical element 250 has a substantially constant diameter that fits inside the internal diameter of introducer 110. In the collapsed state of expandable device 230, distal end 254 of conical element 250 has a smaller or equal diameter to the diameter of proximal end 252 of conical element 250. When expandable device 230 is expanded, distal end 254 of conical element 250 has a larger diameter than the internal diameter of introducer 110.

As depicted in an exemplary embodiment in FIG. 6, conical element 250 includes on the distal section of conical element 250, typically, the majority of the distal section of conical element 250, a preconfigured number of elastic flaps 256 that are separated by a slot cut into the distal section of conical element 250. Flaps 256 can operatively hingedly bend or deflect outwards as a result of pressure being built inside distal section 234 of balloon 230, thus enabling distal section 234 of balloon 230 to expand to the preconfigured volume, distally outside of the typically sharp tip of introducer 110.

Preferably, gas (preferably air) or fluid is injected through cannula 120 and into balloon 230, thereby inflating balloon 230. Hence, proximal section 232 of balloon 230 stretches spring 240 distally, towards distal end 112 of introducer 110, and outside of distal end 112 of introducer 110. As fluid pressure continues to build up, balloon further inflates to an extent that forces flaps 256 of conical element 250 to open, thus facilitating distal section 234 of balloon 230 to reach the preconfigured volume and surface area. Flaps 256 of conical element 250 protect distal section 234 of balloon 230 from being punctured by tip 112 of introducer 110.

Upon deflation of balloon 230, elastic flaps 256 apply the force, stored while being inflated, against distal section 234 of balloon 230, thus collapsing distal section 234 of balloon 230 back into the smaller diameter of the contracted state. As the gas or fluid pressure inside balloon 230 continues to drop, balloon 230 further deflates and spring 240 returns to the unbiased position. Thereby, expandable device 230 is back inside introducer 110, proximal to distal end 112, as depicted in FIG. 5.

When the expandable device is expandable device 230, the expanding-mechanism, typically, includes a pump (not shown) for controllably pump gas (air) or fluid into and out of balloon 230, wherein the operation of the pump is synchronized with biasing element 240 and flaps 256.

An aspect of the present invention is to facilitate determining the tissue type, as well as transition between tissues and cavities. The following describes a method describes the steps of an Epidural anesthesia procedure, by way of example, without limiting the scope of this invention. During Epidural anesthesia procedure, a physician performs procedural steps, in order to differentiate between Ligamentum Flavum 60 and Epidural space 70, wherein a principle intention of the Epidural block procedure is to be able to stop the advancement of introducer 110 upon entering Epidural space 70.

In an embodiment of the present invention, during the inflation of balloon 130, a measurement that reflects the mechanical resistance of the tissue to the inflation of balloon 130, is taken, using selected sensors 170 (see FIG. 3) disposed in pre configured location, within anatomical-positioning apparatus 100. Such sensors can be force sensors, pressure sensors, displacement sensors, strain gauges, tactile sensors, volume sensors, flow sensors, piezoelectric sensors and any other sensors known in the art. The data collected by sensors 170 are sensed while expandable device 130 exerts pressure onto portions of the mammalian tissue. The sensed data is of physical parameters associated with expandable device 130, and reflects mechanical properties of the tissue in which tissue expandable device 130 is disposed while obtaining the sensed data.

The measured physical parameters, associated with expandable device 130, may include the pressure inside balloon 130, the instantaneous volume of balloon 130, the displacement of expandable device 130, the external force applied on the expandable device by the mammalian tissue, spatial force or spatial pressure applied on expandable device 130 or any other measurement known in the art. Other data can be concluded by processing the sensed data using a processor 160, such as the volume-pressure work performed by expandable device 130, volume-pressure profile of expandable device 130, work performed by expandable device 130 and force-displacement profile of expandable device 130, while expandable device exerts pressure onto portions of a mammalian tissue, For example, a pressure sensor can measure the pressure that builds up inside balloon 130 for a given volume. According to the measurement, the type of tissue being pushed by balloon 130 is determined and a decision is made whether to advance introducer 110 by another increment or whether to stop introducer 110 from further advancing, due to entrance into Epidural space 70.

The measurement may be continuous during the expansion, the contraction or the steady expanded state of expandable device 130, thus providing a unique set of measurements for a specific tissue. For instance, a measurement of the external force applied by a mammalian tissue against an expanded expandable device 130 as a function of the displacement of expandable device 130, may provide a curve that is typical for a certain type of tissue, for example an elastic tissue, and is substantially different from a non-elastic tissue.

The measurement can be of spatial force or spatial pressure applied on expandable device 130 by a mammalian tissue. In one embodiment, tactile sensors 175 disposed on expandable device 130 provide spatial data reflecting the mechanical resistance of the tissue being pressed by expandable device 130 in various locations along the circumference of expandable device 130. Such measurements can facilitate determining the direction of the fibers of the mammalian tissue being pressed by the expandable device 130. In addition, such measurements can facilitate determining the existence and direction of a hard tissue (such as a spinal process) that is in the vicinity of the mammalian tissue being pressed by the expandable device 130.

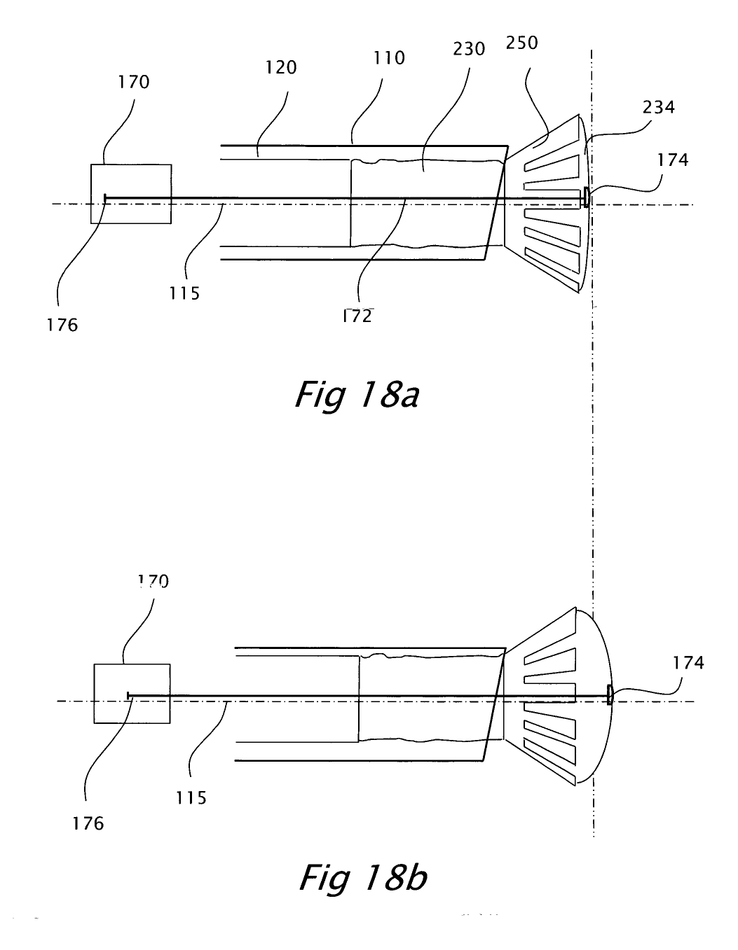

FIG. 18a-18b illustrate an embodiment of the present invention, wherein a distal tip 174 of a guide-wire 172 is attached to distal end 234 of balloon 230, and proximal tip 176 of guide-wire 172 is located near a displacement sensor 170. Upon expansion of balloon 230, distal end 234 of balloon 230 advances forward along longitudinal axis 115, thereby facilitating the advancement of guide-wire 172 along longitudinal axis 115. Displacement sensor 170, for example a photoelectric sensor, measures the displacement of proximal end 176 of guide-wire 172, thereby facilitating determining the displacement of balloon 230 while exerting pressure on portions of a mammalian tissue.

The main advantage of the method for determining tissue type and transition between tissues and cavities by an expandable device is in actively acquiring data regarding a mammalian tissue, by deforming the tissue, in order to determine mechanical properties, such as elasticity, that can otherwise be hard to discover by a passive measurement. Moreover, the larger surface area of distal end 132 of balloon 130, compared the to the tip surface area of tip 112 of introducer 110, finds an expression in that the mechanical resistance of elastic tissues is being amplified by the larger surface area of distal end 132, whereas the mechanical resistance of loose tissues does not change significantly. Thus, it is possible to differentiate between tissues types, although there are variations in population.

In addition, when measuring forces that develop against an introducer 110, friction forces operating on the body of introducer 110 have major influence on the measurement. However, when measuring forces against an expandable device 130, the friction forces operating on the body of introducer 110 have no effect on the measurement. Therefore, a measurement of mechanical resistance against an expandable device is less biased than a measurement against introducer 110.

One more advantage is in the ability to perform multiple measurement by expandable device 130 whether in a specific anatomical location or in a series of locations along the path of tip 112 of introducer 110.

Indicator 180 indicates the type of tissue being in contact with expandable device 130, as well as possible transition between different tissues and cavities. Such an indication can be visual, for example using a display showing a curve of force versus displacement of expandable device, light indication, acoustic indication, for instance when expandable device reaches Epidural space, a gage and a tactile indication, for example a vibration that is indicative to the extent of tissue elasticity.

Referring back to FIGS. 4a-4e, cross sectional views of the distal portion of anatomical-positioning apparatus 100 are illustrated, showing the stages of an Epidural anesthesia procedure, including the penetration of Ligamentum Flavum 60 and including entering into Epidural space 70. In FIG. 4a, Ligamentum Flavum 60 is shown in an undistorted position, as introducer 110 has not touched Ligamentum Flavum 60 as yet. In FIG. 4b, introducer 110 has started to penetrate Ligamentum Flavum 60 and thereby, Ligamentum Flavum 60 is not in an undistorted position, the elasticity of the fibers of Ligamentum Flavum 60 cause Ligamentum Flavum 60 to bend into Epidural space 70 (see concavity 62 in FIGS. 4b and 4c).

As the operator does not see and thereby does not know that introducer 110 has penetrated Ligamentum Flavum 60, the operator activates expanding mechanism 150 to thereby expand balloon 130. The elasticity of Ligamentum Flavum 60 responds to expanded balloon 130, disposed inside Ligamentum Flavum 60, to set a pressure level P₁ inside balloon 130. Knowing, for example the quantity of gas/fluid inserted by expanding mechanism 150, processor 160 analyzes measured pressure P₁, to thereby provide the type of tissue balloon 130 is disposed in. In the described example, processor 160 activates indicator 180 to indicate to the operator that balloon 130 is disposed inside Ligamentum Flavum 60.

Typically, the operator of anatomical-positioning apparatus 100 activates expanding mechanism 150 to bring back expand balloon 130 to contracted state. Alternatively, balloon 130 automatically contracts back to a contracted state. Then, the operator activates an incremental mechanism for controllably advancing both introducer 110 and cannula 120 further towards Epidural space 70. The operator may activate an introducer-incremental-advancing-mechanism 190 (see FIG. 3) and/or a cannula-incremental-advancing-mechanism 192. As the incremental advancing is repeatably performed by the operator, introducer 110 ruptures Ligamentum Flavum 60 penetrates into Epidural space 70, as depicted in FIG. 4d. Introducer-incremental-advancing-mechanism and cannula-incremental-advancing-mechanism 192 are shown in FIG. 3 schematically. The incremental advancing mechanism may be embodied in ways know in the art, such as a screw based mechanism having threads with a preconfigured pitch of 0.5-2 mm, a step motor etc.

Since the anatomical-positioning apparatus 100 is held by the operator's hands and is inserted into a living body, movements of both the operator and the patient may interfere with positioning of tip 112 of introducer 110 inside a mammalian tissue. Therefore, when using the incremental advancing mechanism, the anatomical-positioning apparatus 100 may be stabilized by securing housing 140 of apparatus 100 against the entrance site of introducer 110, for instance, the back of a patient.

When the operator activates expanding mechanism 150 to thereby expand balloon 130, the fat tissue inside Epidural space 70 responds to expanded balloon 130 to set a pressure level P₂ (see FIG. 4e) inside balloon 130. Knowing, for example the quantity of gas/fluid inserted by expanding mechanism 150, processor 160 analyzes measured pressure P₂, to thereby provide the type of tissue balloon 130 is disposed in. In the described example, processor 160 activates indicator 180 to indicate to the operator that balloon 130 is disposed inside Epidural space 70.

### An Exemplary Experiment:

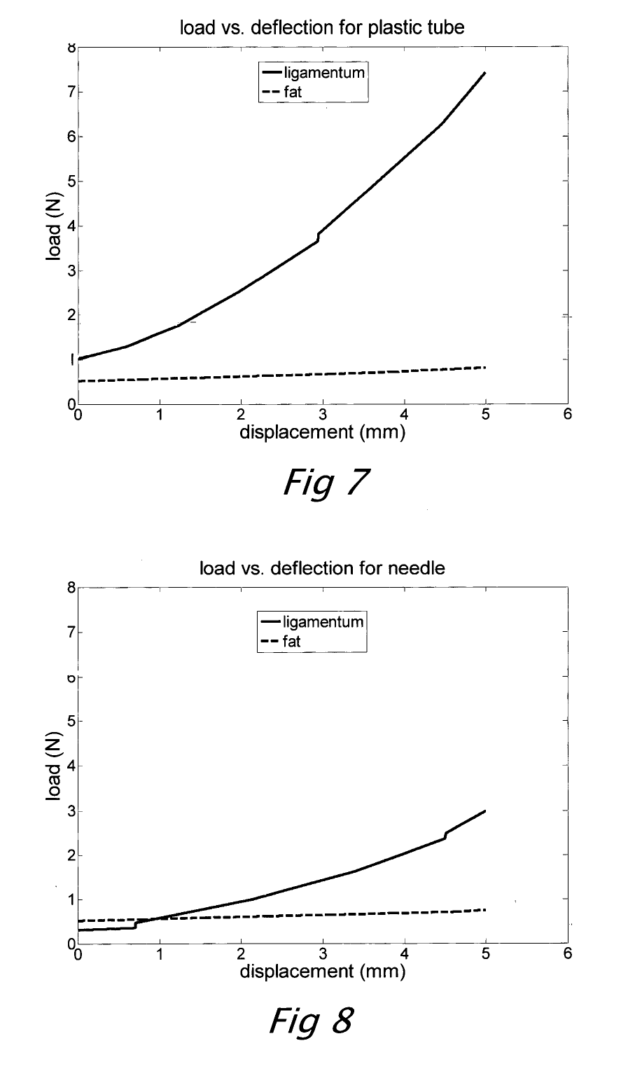

In FIG. 7, the graph describes the resistance forces being measured when advancing a plastic tube with a surface area of ˜4 mm² against a Ligamentum and against fat (wherein the fat simulates the Epidural fat tissue in Epidural space 70). The plastic tube simulates the blunt surface of balloon 130, having a larger surface area than a needle tip. In FIG. 8, the graph describes the resistance forces being measured when advancing a “Tuohy” 18 G needle against a Ligamentum and against fat. The graphs demonstrate a substantial bigger resistance forces measured in the Ligamentum, when advancing a plastic tube, compared to advancing the needle. However, no significant difference in the resistance forces was measured when advancing the plastic tube or the needle against fat tissue. Therefore, the method of measuring forces against an expandable device holds some major advantages and facilitates setting a threshold for differentiating between tissues with different mechanical properties.

Therefore, it is clear that displacing an elastic tissue increases its mechanical resistance, thereby increasing the pressure measured inside a balloon 130, while the resistance of a non elastic and low density tissue remains substantially unchanged under similar displacement. Hence, active measurement of the resistance of a tissue, while expanding or contracting balloon 130 provides data that facilitate determining the type of tissue balloon 130 is situated in.

In variation of the present invention, a ratio between the previous measurement and current measurement is calculated. If the ratio is smaller than a preconfigured threshold value, it is determined that balloon 130 is still inside the Ligamentum Flavum 60, and introducer 110 is incrementally advanced. If the ratio is bigger than the preconfigured value, it means that introducer 110 is inside Epidural space 70. Balloon 130 is deflated and cannula 120 is removed from anatomical-positioning apparatus 100 to enable threading of a catheter through introducer 110 to administrate anesthetics.

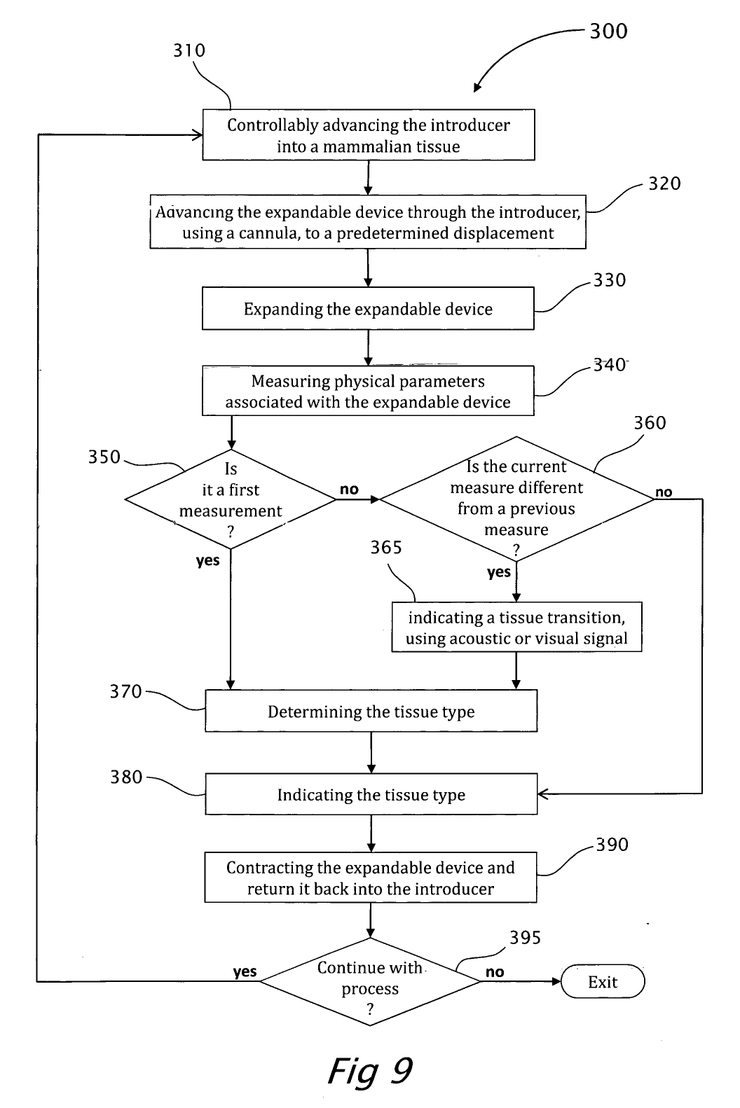

Reference is now made to FIG. 9, a schematic flow chart, showing an exemplary method 300 of determining the tissue type and transition between tissues and cavities, according to embodiments of the present invention. Method 300 begins by administrating introducer 110 of anatomical-positioning apparatus 100 into a mammalian tissue. Method 300 proceeds with the following steps:
Step 310: controllably advancing introducer 110 into a mammalian tissue.
   The operator of anatomical-positioning apparatus 100 controllably advances introducer 110 inside a mammalian tissue.
Step 320: advancing expandable device 130 through introducer 110, using cannula 120, to a predetermined displacement.
   The operator advances expandable device 130 through introducer 110, using cannula 120, to a predetermined displacement. Preferably, the operator uses a cannula-incremental-advancing-mechanism 192, to controllably advance expandable device 130.
Step 330: expanding expandable device 130.
   Expandable device 130 is popped out of introducer 110 and expanded, typically by pressurized flow of gas (typically, air) or fluid, performed by expanding mechanism 150.
Step 340: measuring physical parameters associated with expandable device 130.
   Typically, the pressure inside expandable device 130 is measured as a function of the volume of gas/fluid flown into expandable device 130, and/or the external force applied on expandable device as a function of the displacement of expandable device, and/or any other physical feature being measured during expansion, contraction or expanded steady state of expandable device 130.
Step 350: check if it is the first measuring for the current tissue type.
   If it is the first measuring for the current tissue type, go to step 370.
   Else, preferably, compare current measurement with a previous measurement, typically, the last measurement.
Step 360: check, preferably by processor 160, if the current measurement differs from a previous measure.
   If the current measurement is substantially the same (within a preconfigured threshold value) as the previous measurement, go to step 380.
   Else, it is likely that the current measurement was measured in a different tissue type, compared to the tissue type in which the previous measurement was taken.
Step 365: indicating a tissue transition.
   Indicator 180 indicates a tissue transition event, using an acoustic, visual or any other indicatory signal.
Step 370: determining the tissue type.
   Determine, preferably by processor 160, the tissue type, for example, by comparing the measured physical parameters to a database.
Step 380: indicating the tissue type.
   Indicator 180 indicates a tissue type to the operator.
Step 390: contracting expandable device 130.
   Expandable device 130 is contracted by expanding mechanism 150, typically by evacuating the gas or fluid from within expandable device 130, and thereby, the contracted expandable device 130 also returns back into introducer 110.
Step 395: check if procedure is complete.
   If the operator requests the termination of the process, terminate process.
   Else, go to step 310.

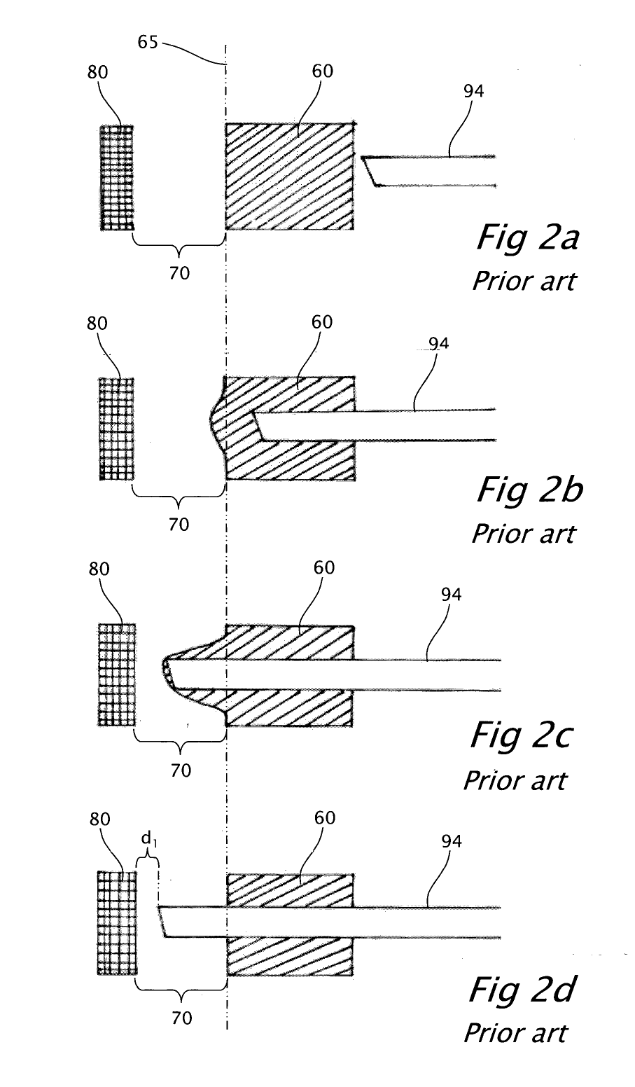

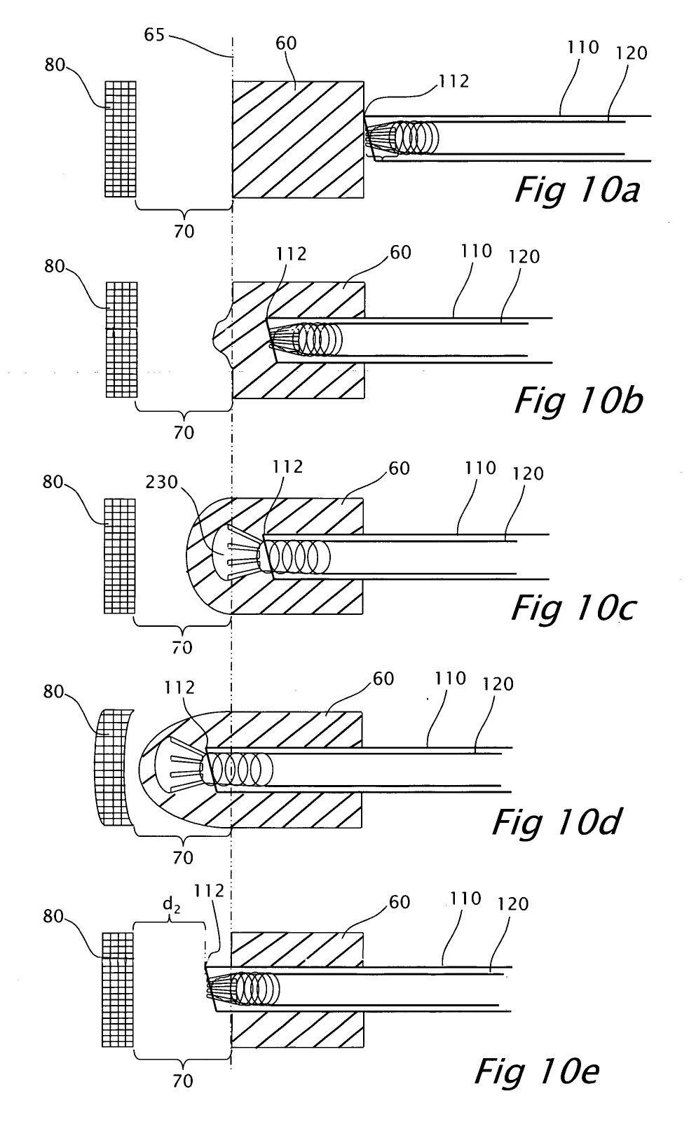

An aspect of the present invention is to provide a method of advancing introducer 110 accurately to a preconfigured in-vivo location, for example, into Epidural space 70, in an Epidural anesthesia (block) procedure. Since, Overshooting of tip 112 of introducer 110 beyond Epidural space 70 may puncture Dura mater 80, it would be advantageous to have a procedure that substantially reduce the risk of tip 112 of introducer 110 puncturing Dura mater 80. It is the intention of the present invention to provide a spring-back method of erupting Ligamentum Flavum 60, such that introducer 110 is inside Epidural space 70 at a distance d₂ (see FIG. 10e) from Dura mater 80, wherein distance d₂ is substantially larger than distance d₁ (see FIG. 2d): d₂>>d₁. The spring-back method is designed for penetrating elastic tissues, and takes advantage of the elasticity of that tissue.

The principle idea behind the various spring-back methods is to bring tip 112 of introducer 110 to a special position beyond virtual line 65, denoting the undistorted position of the wall of Ligamentum Flavum 60 that is the internal wall of Epidural space 70; keep introducer 110 spatially stationary; use expandable device 130 of anatomical-positioning apparatus 100 to further push elastic Ligamentum Flavum 60 by a preconfigured distance; contract expandable device 130 at once and return expandable device 130 back inside introducer 110. Thereby, the elasticity of Ligamentum Flavum 60 moves Ligamentum Flavum 60 towards the undistorted position 65, rapidly. Thereby, spearingly meeting tip 112 of introducer 110, on the way back, which cause Ligamentum Flavum 60 to rupture after one or more spring-back iterations.

In FIGS. 10a-10e, a spring-back method, according to embodiments of the present invention, for puncturing, for example, Ligamentum Flavum 60 using, for example, expandable device 230 of anatomical-positioning apparatus 200 (FIGS. 5 and 6), is illustrated. The spring-back method is described through the Epidural block procedure, without limiting the scope of this invention.

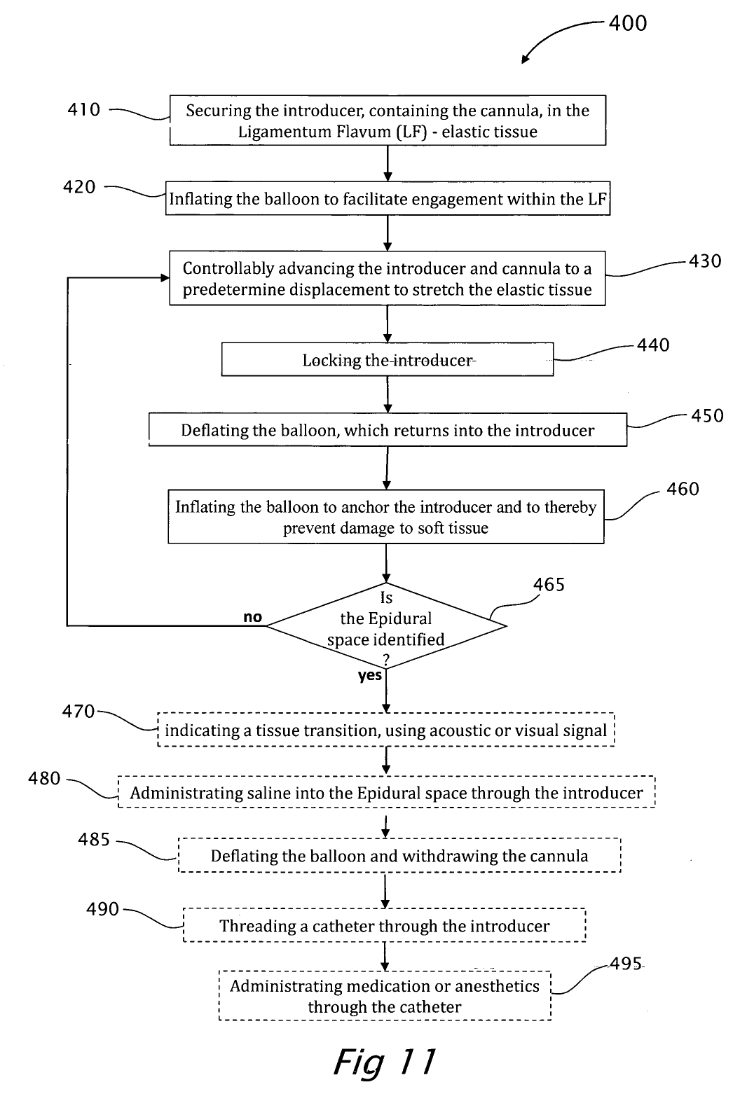

Reference is now made to FIG. 11, a schematic flow chart, showing an exemplary method 400 of safely advancing an introducer 110 into Epidural space 70 then further administrating medication or anesthetics through a catheter into Epidural space 70, during an Epidural block procedure, according to embodiments of the present invention. Method 400 begins by administrating introducer 110 of anatomical-positioning apparatus 200 into a mammalian tissue, as shown in FIG. 10a. Method 400 proceeds with the following steps:
Step 410: securing introducer 110, containing cannula 120, in Ligamentum Flavum 60.
   The operator of anatomical-positioning apparatus 200 secures introducer 110, containing cannula 120, in Ligamentum Flavum 60, Ligamentum Flavum 60 being an elastic tissue (See FIG. 10b). The elastic tissue stretches slightly beyond the original, undistorted position 65. Introducer 110 is advanced into Ligamentum Flavum 60 after departing from Interspinous Ligament 52.
Step 420: inflating balloon 230 to facilitate engagement with the fibers of Ligamentum Flavum 60.
   The operator activates expandable mechanism 150 and thereby, expandable device 230 is disposed out of introducer 110 and distal section 234 of balloon 230 expanded, typically by pressurized flow of gas (typically, air) or fluid, performed by expanding mechanism 150. Distal section 234 of balloon 230 expands against flaps 256 of conical element 250, causing flaps 256 to open outwardly, thereby protecting balloon 230 from being punctured by tip 112 of introducer 110 (FIG. 10c).
Step 430: controllably advancing introducer 110 and cannula 120 to a predetermine displacement to stretch the elastic tissue (Ligamentum Flavum 60).
   The operator advances introducer 110 together with cannula 120 and expandable device 230 using introducer-incremental-advancing-mechanism 190 and cannula-incremental advancing mechanism 192 respectively, to a predetermined displacement (See FIG. 10d), while expandable device 230 is steadily expanded. Thereby, elastic tissue stretches extensively beyond the original, undistorted position 65 of the elastic tissue wall.
Step 440: locking introducer 110.
   Locking introducer 110 in position, using introducer-incremental-advancing-mechanism 190.
Step 450: deflating balloon 230, which returns into introducer 110.
   The operator rapidly deflates expandable device 230 to facilitate a rapid return of balloon 230 into introducer 110. In a preferred embodiment, conical element 250 and spring 240 facilitate the rapid return on balloon 230 into introducer 110.
   Ligamentum Flavum 60 rapidly returns to the undistorted position, towards and possibly over introducer 110. If tip 112 of introducer 110 is disposed beyond the original, undistorted position 65 of Ligamentum Flavum 60, introducer 110 penetrates Ligamentum Flavum 60 at a predetermined location.
Step 460: inflating balloon 230 to anchor introducer 110 and to thereby prevent damage to soft tissue.
   Balloon 230 is inflated to anchor introducer 110 and to thereby prevent introducer 110 from advancing forward towards soft tissues, thus protecting them. For example, if introducer is inside Epidural space, there is a need for protecting Dura mater 80 or other anatomical structures inside Epidural space such as blood vessels, from being punctured by introducer 110.
Step 465: check if Epidural space 70 identified.
   Follow the procedure, such as method 300, to identify the type of tissue. If the fat tissue of Epidural space 70 is not identified, but rather that Ligamentum Flavum 60 is still identified, go to step 430.
   Else, optionally, proceed with the Epidural block procedure.
Step 470: indicating a tissue transition, using acoustic or visual signal.
   Indicator 180 indicates a tissue transition event (from Ligamentum Flavum 60 to Epidural space 70), using an acoustic, visual or any other indicatory signal.
Step 480: administrating saline into Epidural space 70 through introducer 110.
   Administrate saline into Epidural space 70 through introducer 110 to thereby locally enlarge Epidural space 70 to facilitate insertion of a catheter.
Step 485: deflating balloon 230 and withdrawing cannula 120.
   Deflate balloon 230 and withdraw cannula 120 to facilitate insertion of a catheter.
Step 490: threading a catheter through introducer 110.
   A catheter is inserted to. Epidural space 70 through introducer 110.
Step 495: administrating medication or anesthetics through the catheter.
   Administrate medication or anesthetics through the catheter.

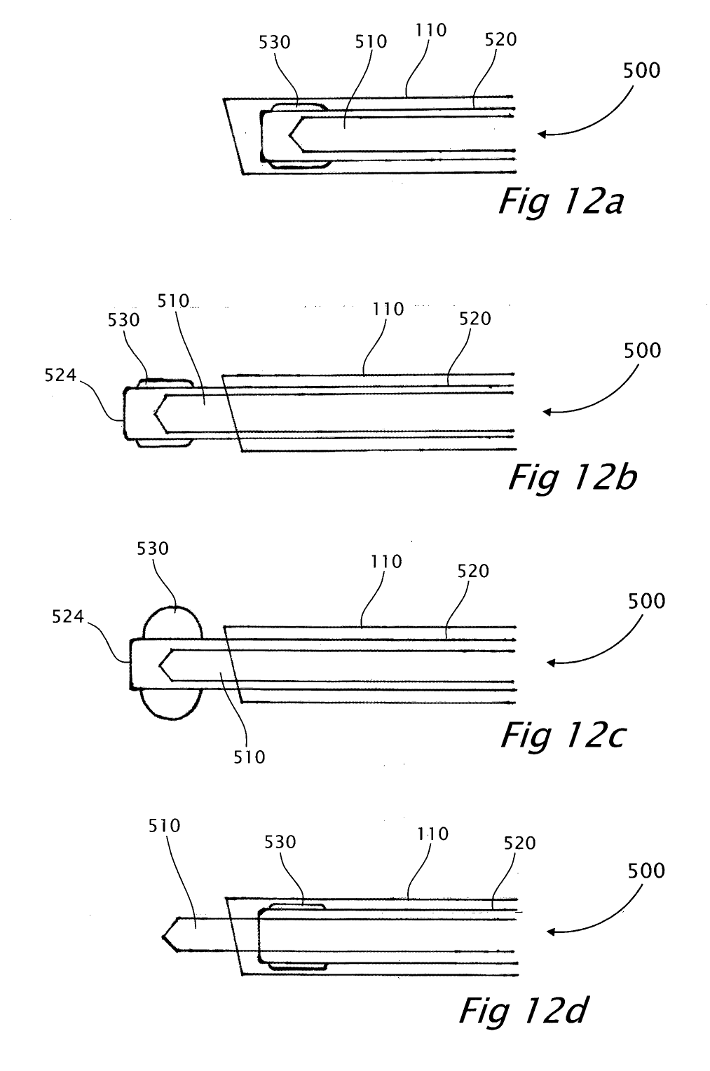

In FIGS. 12a-12d, another anatomical-positioning apparatus 500 facilitating a spring-back method, according to other embodiments of the present invention, for puncturing, for example, Ligamentum Flavum 60 using, for example, expandable device 530 of anatomical-positioning apparatus 500, is illustrated. The spring-back method is described through the Epidural block procedure, without limiting the scope of this invention. Anatomical-positioning apparatus 500 includes a cannula 520, typically a double lumen cannula, and a thin-needle 510 facilitated to incrementally advance with respect to cannula 520. The expandable device includes a balloon 530 disposed on the external circumference of cannula 520, proximal to distal end 524 of cannula 520.

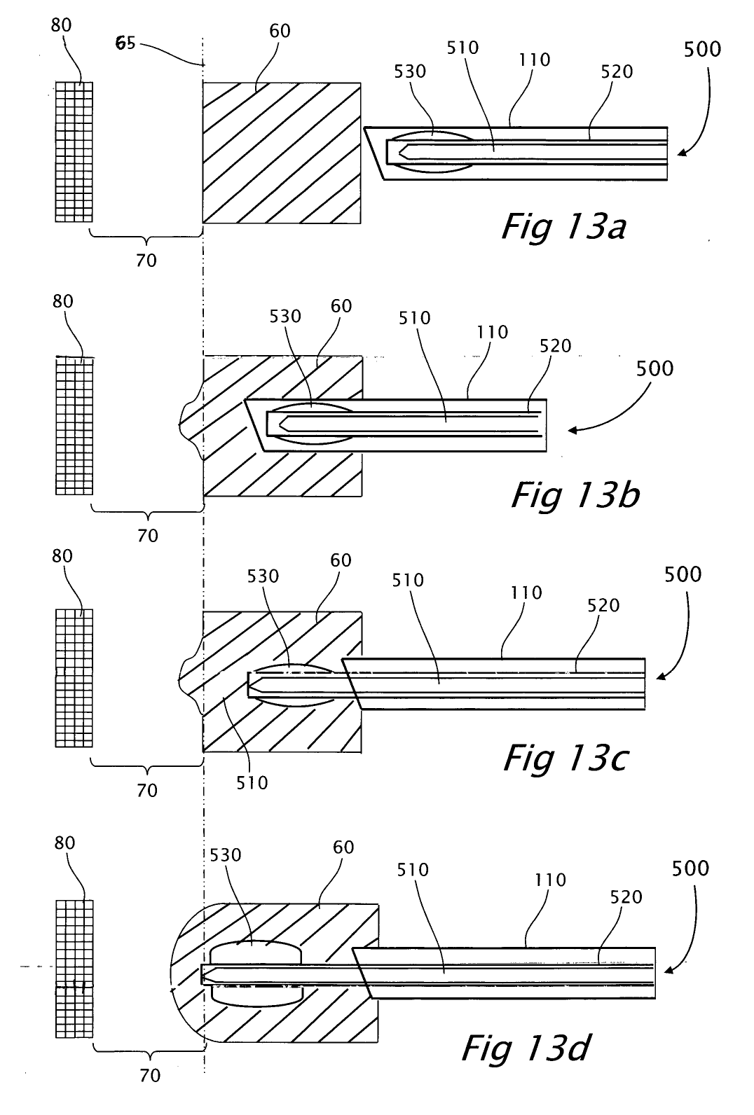

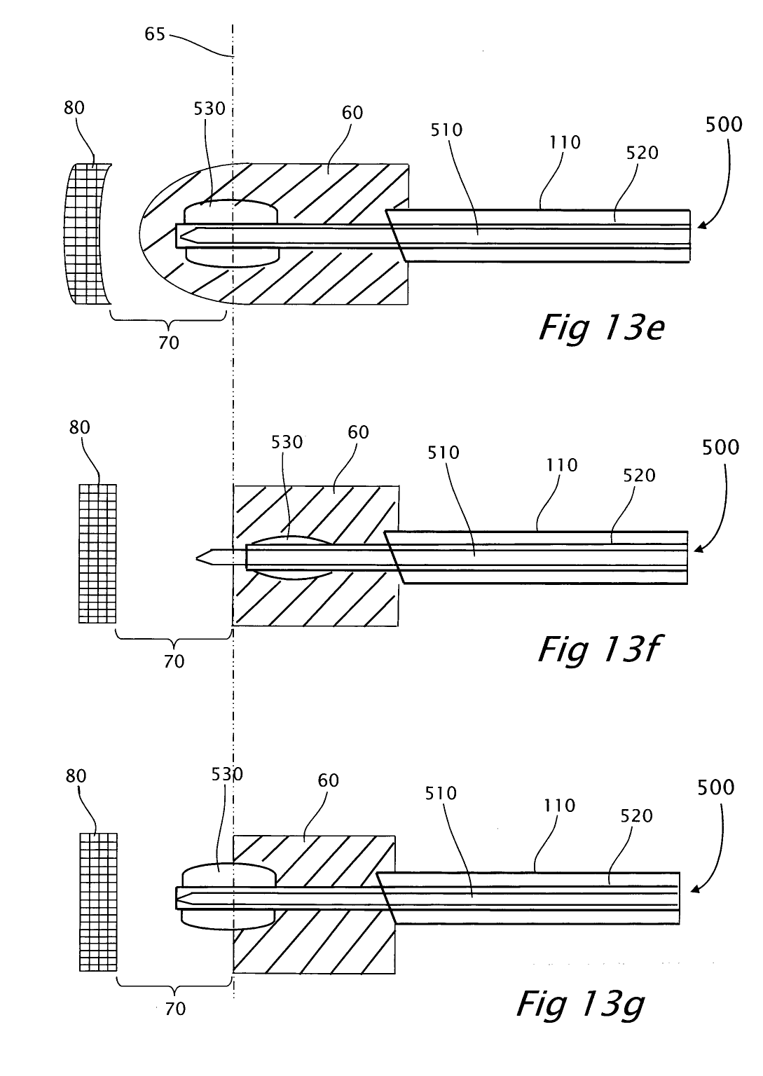

FIGS. 13a-13g illustrate cross sectional views of the distal portion of anatomical-positioning apparatus 500, showing the stages of a spring-back method of safe penetration of an elastic tissue, for example, Ligamentum Flavum 60 using, for example, expandable device 530 of anatomical-positioning apparatus 500, is illustrated.

In this spring-back method, when the elasticity of Ligamentum Flavum 60 rapidly moves Ligamentum Flavum 60 back towards the undistorted position 65, Ligamentum Flavum 60 is spearingly meeting the tip of thin-needle 510, rather than tip 112 of introducer 110. Eventually, thin-needle 510 causes Ligamentum Flavum 60 to rupture after one or more spring-back iterations.

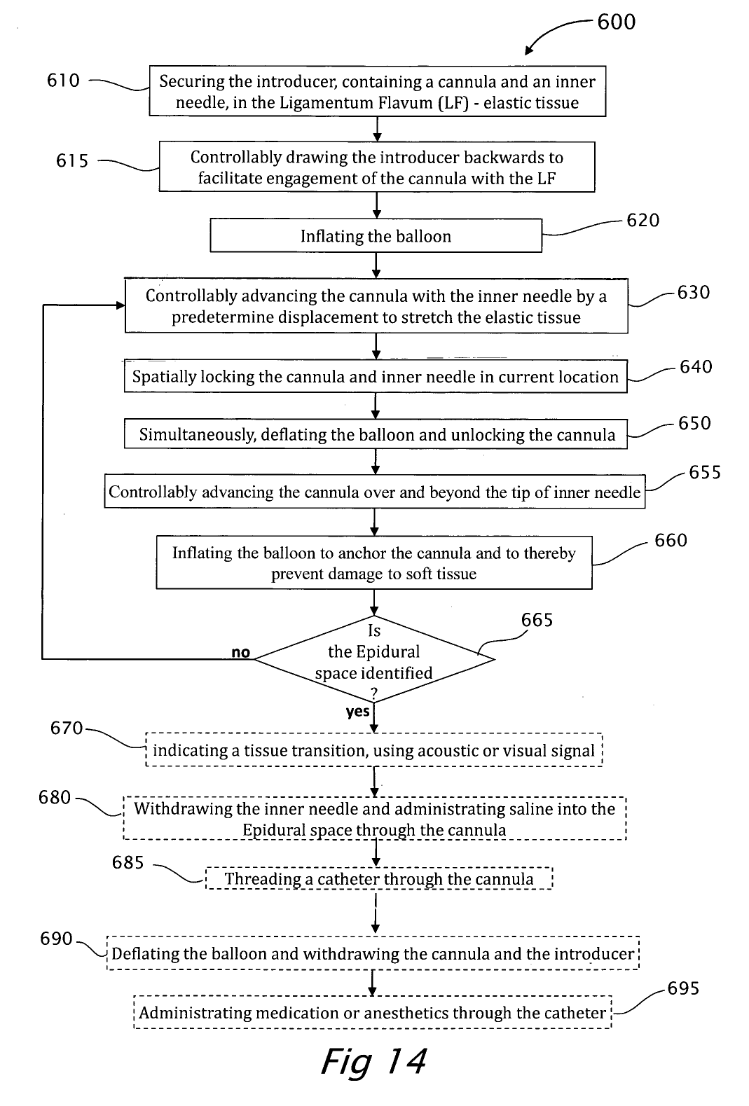

Reference is now made to FIG. 14, a schematic flow chart, showing an exemplary method 600 of safely advancing an inner needle 510 and a cannula 520 into Epidural space 70 then further administrating medication or anesthetics through the catheter into

Epidural space 70, during an Epidural block procedure, according to embodiments of the present invention. Method 600 begins by administrating introducer 110 of anatomical-positioning apparatus 500 into a mammalian tissue (See FIG. 13a). Method 600 proceeds with the following steps:
Step 610: securing introducer 110, containing cannula 520 and inner needle 510 in Ligamentum Flavum 60.
   The operator of anatomical-positioning apparatus 500 secures introducer 110, containing cannula 520 and inner needle 510, in. Ligamentum Flavum 60, Ligamentum Flavum 60 being an elastic tissue (See FIG. 13b). The elastic tissue stretches slightly beyond the original, undistorted position 65. Introducer 110 is advanced into Ligamentum Flavum 60 after departing from Interspinous Ligament 52.
Step 615: Controllably drawing introducer 110 backwards, with respect to cannula 520.
   The operator draws introducer 110 backwards, with respect to cannula 520 (see FIG. 13c), to facilitate engagement of cannula 520 and balloon 530, attached thereto, with the fibers of Ligamentum Flavum 60.
Step 620: inflating balloon 530.

The operator activates expandable mechanism 150 and thereby, balloon 530 is expanded (see FIG. 13d), typically by pressurized flow of gas (typically, air) or fluid, performed by expanding mechanism 150. Balloon 530 expands against the fibers of Ligamentum Flavum 60.
Step 630: controllably advancing cannula 520 and inner needle 510 by a predetermine displacement to stretch the elastic tissue (Ligamentum Flavum 60).
   The operator advances cannula 520 (together with the expanded balloon 530) and inner needle 510 by a predetermined displacement (see FIG. 13e). Thereby, elastic tissue stretches extensively beyond the original, undistorted position 65.
Step 640: spatially locking cannula 520 and inner needle 510 in current location.

Locking cannula 520 and inner needle 510 in position, using inner needle-incremental-advancing-mechanism and cannula-incremental-advancing-mechanism 192.
Step 650: simultaneously, deflating balloon 530 and unlocking cannula 520.
   Simultaneously, deflating balloon 530 and unlocking cannula 520.
   Ligamentum Flavum 60 rapidly returns to the undistorted position, towards and possibly over inner needle 510. If the tip of inner needle 510 is disposed beyond the original, undistorted position 65 of Ligamentum Flavum 60, inner needle 510 penetrates Ligamentum Flavum 60 at a predetermined location (see FIG. 13f).
Step 655: controllably advancing cannula 520 over and beyond the tip of inner needle 510.
   The operator advances cannula 520 over and beyond the tip of inner needle 510, wherein cannula 520 serves both as a sheath for inner needle 510 to protect the immediate surroundings of inner needle 510 (see FIG. 13g) and as a pathway for the insertion of a catheter.
Step 660: inflating balloon 530 to anchor cannula 520 and to thereby prevent damage to soft tissue.
   Balloon 530 is inflated to anchor cannula 520 and to thereby prevent damage to soft tissue (see FIG. 13g). Should distal end 524 of cannula 520 have reached Epidural space 70, prevent unwarranted motion of cannula 520 inside Epidural space 70.
Step 665: check if Epidural space 70 identified.
   Follow the procedure, such as method 300, to identify the type of tissue. If the fat tissue of Epidural space 70 is not identified, but rather that Ligamentum Flavum 60 is still identified, go to step 630.
   Else, optionally, proceed with the Epidural block procedure.
Step 670: indicating a tissue transition, using acoustic or visual signal.
   Indicator 180 indicates a tissue transition event (from Ligamentum Flavum 60 to Epidural space 70), using an acoustic, visual or any other indicatory signal.
Step 680: Withdrawing inner needle 510 and administrating saline into Epidural space 70 through cannula 520.
   Withdraw inner needle 510 to facilitate a pathway for administrating saline into Epidural space 70 through cannula 520 to thereby locally enlarge Epidural space 70 to facilitate insertion of a catheter.
Step 685: Threading a catheter through cannula 520.
   A catheter is inserted to Epidural space 70 through cannula 520.
Step 690: deflating balloon 530 and withdrawing cannula 520 and introducer 110.
   Deflating balloon 530 and withdrawing cannula 520 and introducer 110, so that only a catheter is placed inside Epidural space 70.
Step 695: administrating medication or anesthetics through the catheter.
   Administrate medication or anesthetics through the catheter.

An aspect of the present invention is to provide an anatomical-positioning apparatus and method of use thereof, having an introducer with an openable tip.

The principle idea behind an introducer with an openable tip is to have a balloon trapped inside the openable tip. The openable tip of the introducer has a closed state and an opened state, wherein in the closed state the openable tip forms a sharp tip facilitating penetration in and through a tissue, and wherein in the opened state the openable tip expands to forms a blunt surface, anchoring the introducer in position.

A preconfigured pressure is maintained inside the balloon trapped inside the openable tip. As long as introducer with a openable tip is situated inside a tissue, such as

Ligamentum Flavum 60, wherein the pressure applied on the openable tip by the tissue exceeds the preconfigured pressure inside the balloon, the openable tip stays in a closed state. Once the openable tip of the introducer moves into a softer tissue, such as in Epidural space 70, wherein the surrounding pressure, due to tissue properties, is significantly lower than the internal pressure inside the balloon, the balloon expands rapidly. Thereby, the expanding balloon causes the openable tip of the introducer to open and anchor the openable tip of the introducer inside Epidural space 70, wherein the balloon protects tissues and anatomical structures ahead of it.

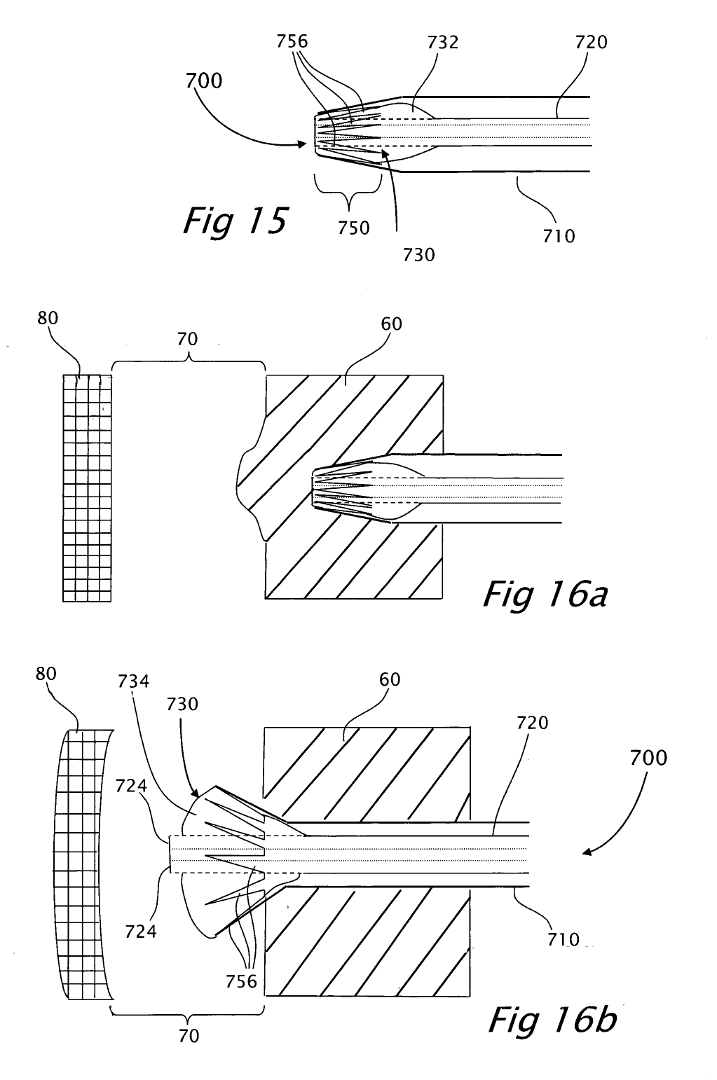

FIGS. 15, 16a and 16b, illustrate the distal section of an anatomical-positioning apparatus 700, according to variations of the present invention, includes an introducer 710 having an openable tip 750, for puncturing a tissue, for example, Ligamentum Flavum 60. Anatomical-positioning apparatus 700 is described through the Epidural block procedure, without limiting the scope of this invention. FIG. 15 illustrates openable tip 750 in a closed state. FIG. 16a illustrates openable tip 750 in a closed state penetrates through a tissue, such as Ligamentum Flavum 60. FIG. 16b illustrates openable tip 750 in an opened state after penetrating into a softer tissue, such as Epidural space 70.

Anatomical positioning apparatus 700 further includes cannula 720 having an expandable device 730, typically a balloon, wherein proximal end 732 of balloon 730 is securely attached to the external circumference of cannula 720, proximal to distal end 724 of cannula 720. Openable tip 750 includes a preconfigured number of elastic flaps 756 that are separated by a slot cut into the distal section of flexible tip 750. Typically, flaps 756 have a pointing tip and can operatively hingedly bend or deflect outwards as a result of a preconfigured pressure being built inside balloon 732, when the pressure external to flaps 756 is lower than the pressure inside balloon 732. Otherwise, balloon 730 stays trapped inside openable tip 750 of introducer 710 and thereby, openable tip 750 can be advanced through tissue.

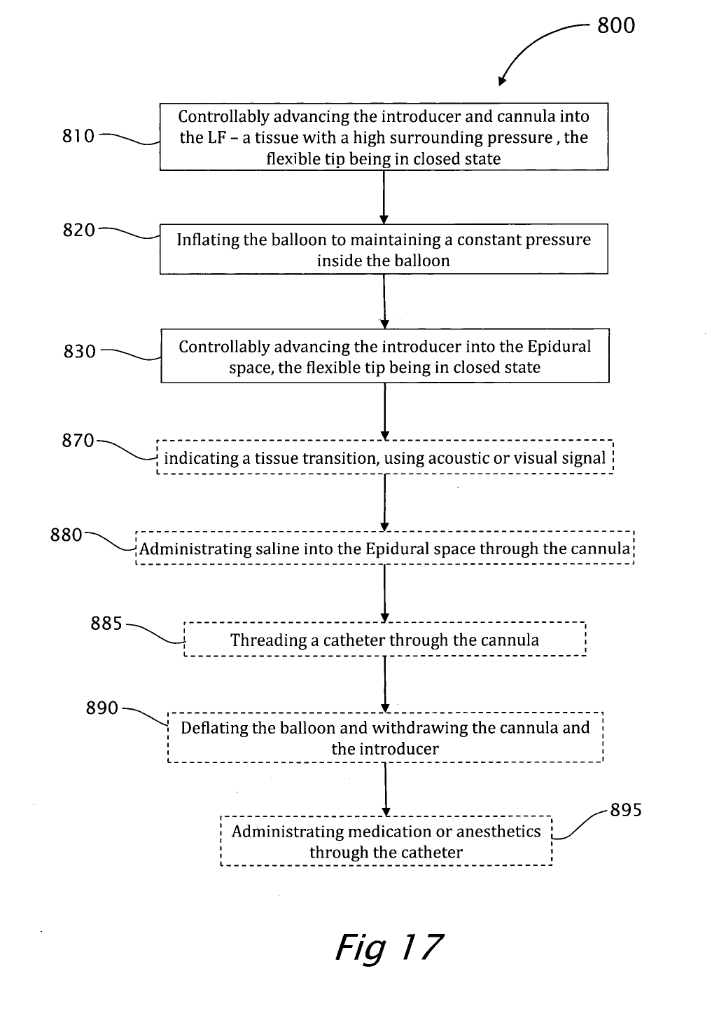

Reference is now made to FIG. 17, a schematic flow chart, showing an exemplary method 800 of administrate medication or anesthetics through the catheter into Epidural space 70, during an Epidural block procedure, according to embodiments of the present invention. Method 800 begins by administrating introducer 710 of anatomical-positioning apparatus 700 into a mammalian tissue wherein openable tip 750 of introducer 710 is in closed state. Method 800 proceeds with the following steps:
Step 810: controllably advancing introducer 710, containing cannula 720, into and inside an elastic tissue.
   The operator advances introducer 710, containing cannula 720, into and inside a soft tissue with high surrounding pressure, such as Ligamentum Flavum 60. Openable tip 750 is in a closed state.
Step 820: inflating balloon 730 to maintaining a constant pressure inside balloon 730.
   The operator activates an expandable mechanism 150 to inflate balloon 730 and to thereby, maintaining a constant preconfigured pressure inside balloon 730. Situated inside a soft tissue, having fibers that exert pressure on the external sides of flaps 756 the pressure being greater than the pressure inside balloon 730, openable tip 750 remain in a closed state.
Step 830: controllably advancing introducer 710 and cannula 720 into Epidural space 70, the openable tip being in closed state.
   The operator further advances introducer 710 and cannula 720, openable tip 750 remaining in closed state. Once openable tip 750 of introducer 710 enters Epidural space 70, the external pressure exerted on the external sides of flaps 756 drops substantially, bellow the preconfigured pressure inside balloon 730. As a result, openable tip 750 opens up into an opened state as balloon 730 expands inside Epidural space 70, thus protecting soft tissues and structures ahead of balloon 730.
Step 870: indicating a tissue transition, using acoustic or visual signal.
   The indicator of anatomical-positioning apparatus 700 indicates a tissue transition event (from Ligamentum Flavum 60 to Epidural space 70), using an acoustic, visual or any other indicatory signal.
Step 880: administrating saline into Epidural space 70 through cannula 720.
   Administrate saline into Epidural space 70 through cannula 720 to thereby locally enlarge Epidural space 70 to facilitate insertion of a catheter.
Step 885: threading a catheter through cannula 720.
   A catheter is inserted to Epidural space 70 through cannula 720.
Step 890: deflating balloon 730 and withdrawing introducer 710 and cannula 720.
   Deflate balloon 730 and withdrawing introducer 710 and cannula 720.
Step 895: administrating medication or anesthetics through the catheter.
   Administrate medication or anesthetics through the catheter.

The invention being thus described in terms of several embodiments and examples, it will be obvious that the same may be varied in many ways. Such variations are not to be regarded as a departure from the spirit and scope of the invention, and all such modifications as would be obvious to one skilled in the art.

What is claimed is:

1. An anatomical-positioning apparatus for acquiring mechanical data from a mammalian tissue to facilitate determining type of said tissue and transition between different tissues and cavities, comprising:
a) a cannula having a tip at the distal section of said cannula;
b) an expandable device having a contracted form size, when in a contracted state, and an expanded form size, when in an expanded state, wherein said expanded form size is substantially larger than said contracted form size;
c) an introducer, having a longitudinal axis and a distal end, facilitating the introduction of said expandable device into said mammalian tissue, said expandable device being in said contracted state;
d) an expanding-mechanism for expanding and contracting said expandable device; and
e) a sensor for measuring physical parameters associated with said expandable device, to thereby provide sensed data, while said expandable device exerts pressure onto portions of said mammalian tissue, to thereby facilitating determining the type of tissue that is in contact with said expandable device and to determine a transition between different tissues and cavities,
wherein said expandable device is securely attached to said tip of said cannula or on the external circumference of said cannula, proximal to said tip of said cannula; and
wherein when said expandable device expands from said contracted state to said expanded form size, said expandable device is disposed outside said introducer, and a blunt contact surface is formed at the external surface of said expanded expandable device.

2. The anatomical-positioning apparatus as in claim 1, wherein said expandable device protects said mammalian tissue or an anatomical structure ahead of said mammalian tissue, from being punctured by said introducer of said expandable device.

3. The anatomical-positioning apparatus as in claim 1, wherein said cannula protects said expandable device from being punctured by said introducer.

4. The anatomical-positioning apparatus as in claim 1, wherein said expandable device expands and contracts under fluid or gas pressure provided by said expanding-mechanism through said cannula.

5. The anatomical-positioning apparatus as in claim 1, wherein said sensor includes one or more sensing devices selected from the group of devices consisting of a pressure sensor, strain gauge, force sensor, tactile sensor, displacement sensor, volume sensor, flow sensor and a piezoelectric transducer.

6. The anatomical-positioning apparatus as in claim 5, wherein said sensing device includes an array of sensors disposed on said expandable device.

7. The anatomical-positioning apparatus as in claim 1, wherein said sensed data is indicated by one or more indication devices selected from the group of devices consisting of a display, a gage, a light indicator, an audio indicator and a tactile indicator.

8. The anatomical-positioning apparatus as in claim 1, wherein said introducer is an openable introducer, having an openable sharp tip; wherein said openable sharp tip of said openable introducer has a closed state and an open state; and wherein said expandable device is trapped inside said openable sharp tip, said openable sharp tip being in a closed state.

9. The anatomical-positioning apparatus as in claim 8, wherein said expandable device facilitates said openable sharp tip to remain in said closed state, when said expandable device is loaded by a preconfigured expanding force and said sharp tip is situated in a high density tissue;
wherein said expandable device facilitates the opening of said openable sharp tip, when said expandable device is loaded be a preconfigured expanding force and said openable sharp tip enters a low density tissue or cavity; wherein said closed state facilitates advancement of said openable introducer through said mammalian tissue; and wherein said open state prevents advancement of said openable introducer through said mammalian tissue.

10. The anatomical-positioning apparatus as in claim 1, wherein said introducer is selected from the group consisting of a veress needle, an epidural needle, a biopsy needle, a trocar, a cannula, a catheter, a Tuohy type needle and a surgical instrument.

11. The anatomical-positioning apparatus as in claim 1, wherein said expandable device is selected from a group of devices consisting of a balloon, a membrane, a diaphragm, a spring and a flexible device.

12. The anatomical-positioning apparatus as in claim 11, wherein said expandable device comprises one or more mechanical devices selected from a group of devices consisting of a spring and a balloon enclosed in a conical element, wherein said conical element protects said balloon from being punctured.

13. The anatomical-positioning apparatus as in claim 1, wherein said cannula and said expandable device is introduced on-demand and can be withdrawn during a procedure in order to enable utilization of other tools inside said mammalian tissue.

14. The anatomical-positioning apparatus as in claim 4, wherein said cannula includes a double lumen, wherein a first lumen facilitates a pathway for said fluid or gas into said expandable device, and wherein a second lumen facilitates a pathway for one or more devices selected from a group of devices consisting of a thin needle, a catheter, an optical fiber, an electrode and a surgical instrument.

15. The anatomical positioning apparatus as in claim 4, wherein said cannula includes a single lumen facilitating a pathway for said fluid or gas into said expandable device, and wherein said single lumen facilitating a pathway for an optical fiber or a miniature camera, to thereby facilitate visualization of said mammalian tissue through said expandable device, said expandable device being in contact with said mammalian tissue.

16. The anatomical-positioning apparatus as in claim 1 further comprises an advancement mechanism for incrementally advancing, in a controlled manner, one or more devices selected from the group consisting of said introducer, said cannula and said expandable device.

17. The anatomical-positioning apparatus as in claim 16, wherein said advancement mechanism is selected from the group of devices consisting of a motor based device, hydraulic device, gear based device and a screw based device.

18. The anatomical-positioning apparatus as in claim 16, wherein said introducer can be advanced incrementally together with said cannula and said expandable device.

19. The anatomical-positioning apparatus as in claim 16, wherein said introducer can be advanced incrementally with respect to said cannula and said expandable device.

20. The anatomical-positioning apparatus as in claim 1 further comprises a processor facilitated to record and analyze said sensed data, to thereby determine said type of tissue that is in contact with said expandable device and to determine said transition between tissues and cavities, wherein said sensed data is acquired while said expandable device is expanding, contracting or in a steady expansion level.

21. The anatomical-positioning apparatus as in claim 1, wherein said sensed data includes a measurement of the advancement of said cannula against said mammalian tissue, and wherein the displacement of said expandable device, along said longitudinal axis, is inferred from said sensed data.

22. The anatomical-positioning apparatus as in claim 1, wherein said sensed data includes a one-dimensional measurement of the displacement of said expandable device, during said expanding against said mammalian tissue or contracting away from said mammalian tissue.

23. The anatomical-positioning apparatus as in claim 1, wherein said sensed data includes one or more measurements selected from a group of measurements consisting of the pressure inside said expandable device, the volume of gas or fluid inside said expandable device, an external force applied on said expandable device by said mammalian tissue and the spatial pressure or spatial force applied on said expandable device by said mammalian tissue, while said expandable device exerts pressure onto portions of said mammalian tissue.

24. The anatomical-positioning apparatus as in claim 20, wherein said sensed data facilitates determining one or more measurements selected from a group of measurements consisting of the volume-pressure work performed by said expandable device, a volume-pressure profile of said expandable device, the work being done by said expandable device and the force-displacement profile of said expandable device, while said expandable device exerts pressure onto portions of said mammalian tissue.

25. A method for acquiring mechanical data from a mammalian tissue by an expandable device, while said expandable device exerts pressure onto portions of said mammalian tissue, the method comprising the steps of:
a) providing an anatomical-positioning apparatus including:
i. a cannula having a tip at the distal section of said cannula;
ii. an expandable device having a contracted form size, when in a contracted state, and an expanded form size, when in an expanded state, wherein said expanded form size is substantially larger than said contracted form size, and wherein said expandable device is securely attached to said tip of said cannula or on the external circumference of said cannula, proximal to said tip of said cannula;
iii. an introducer, having a distal end, facilitating the introduction of said expandable device into said mammalian tissue, said expandable device being in said contracted state;
iv. an expanding-mechanism for expanding and contracting said expandable device; and
v. a measuring-mechanism for measuring physical parameters associated with said expandable device, while said expandable device exerts pressure onto portions of said mammalian tissue;
b) inserting said introducer inside said mammalian tissue, wherein said distal end of said introducer enters first;
c) advancing said expandable device through said introducer, using said cannula;
d) expanding said expandable device outside said distal end of said introducer; and
e) measuring said physical parameters associated with said expandable device, while said expandable device exerts pressure onto portions of said mammalian tissue, thereby creating sensed data.

26. The method as in claim 25 further including the step of analyzing said sensed data, using a processor, thereby determining the type of said mammalian tissue being in contact with said expandable device, and determining a transition between different mammalian tissues and cavities.

27. The method as in claim 25, wherein said sensed data includes one or more measurements selected from a group of measurements consisting of the displacement of said expandable device and cannula, the pressure inside said expandable device, the volume of gas or fluid inside said expandable device, the external force applied on said expandable device by said mammalian tissue and the spatial pressure or spatial force applied on said expandable device by said mammalian tissue, while said expandable device exerts pressure onto portions of said mammalian tissue.

28. The method as in claim 26, wherein said sensed data facilitates determining one or more measurements selected from a group of measurements consisting of the volume-pressure work performed by said expandable device, the volume-pressure profile of said expandable device, the work being done by said expandable device and the force-displacement profile of said expandable device, while said expandable device exerts pressure onto portions of said mammalian tissue.

29. The method as in claim 25, wherein said measuring of said sensed data is performed during states selected from a group of states consisting of an expansion of said expandable device, a contraction of said expandable device and a displacement of said cannula and said expandable device, while said expandable device is expanded.

30. A method for a controlled insertion of an introducer into and through an elastic mammalian tissue, the method comprising the steps of:
a) providing a device including:
i. a cannula having a tip at the distal section of said cannula;
ii. an expandable device having a contracted form size, when in a contracted state, and an expanded form size, when in an expanded state, wherein when in expanded state, said expandable device has a front end; wherein said expanded form size is substantially larger than said contracted form size; and wherein said expandable device is securely attached to said tip of said cannula or on the external circumference of said cannula, proximal to said tip of said cannula;
iii. an introducer, having a sharp tip at the distal end, facilitating the introduction of said expandable device into said mammalian tissue, said expandable device being in said contracted state;
iv. an expanding-mechanism for expanding and contracting said expandable device; and
v. an advancement mechanism for incrementally advancing, in a controlled manner, devices selected from the group consisting of said introducer, said cannula and said expandable device;
b) expanding said expandable device by said expanding-mechanism, outside said tip of said introducer, whereby said expandable device exerts pressure onto portions of said mammalian tissue, wherein said mammalian tissue is initially in an undistorted position;
c) advancing said introducer incrementally to a preconfigured position situated beyond said undistorted position of said mammalian tissue, said mammalian tissue being pushed by said expandable device, wherein said tip of said introducer is posterior to the instantaneous position of said front end of said expandable device, with respect to said elastic tissue;
d) holding said introducer in secured position; and
e) contracting said expandable device at a preconfigured rate, thereby facilitating said mammalian tissue, being elastic, to spring back powerfully towards said undistorted position of said mammalian tissue, thereby penetrating said mammalian tissue by said tip of said introducer, being disposed beyond said undistorted position of said mammalian tissue.

31. The method as in claim 30, wherein said incremental advancing of said introducer is performed while also incrementally advancing said expandable device, wherein said expandable device is expanded.

32. The method as in claim 30, wherein said incremental advancing of said introducer is performed, while said expandable device is expanded and in steady position, wherein said introducer is incrementally displaced beyond said undistorted position of said mammalian tissue, and wherein said introducer tip is kept at a shorter distance from said mammalian tissue, with respect to steady position of said front end of said expandable device.

33. A method for a controlled insertion of a cannula, using an inner needle, into and through an elastic mammalian tissue, the method comprising the steps of:
a) providing a device including:
i. a cannula having a double lumen and a tip at the distal section of said cannula;
ii. an expandable device having a contracted form size, when in a contracted state, and an expanded form size, when in an expanded state, wherein when in expanded state, said expandable device has a front end; wherein said expanded form size is substantially larger than said contracted form size; and wherein said expandable device is securely attached on the external circumference of said cannula, proximal to said tip of said cannula;
iii. an introducer, facilitating the introduction of said expandable device into said mammalian tissue, said expandable device being in said contracted state;
iv. an expanding-mechanism for expanding and contracting said expandable device;
v. an inner needle being introduced through said cannula; and
vi. an advancement mechanism for incrementally advancing, in a controlled manner, devices selected from the group consisting of said introducer, said cannula, said inner needle and said expandable device;
b) Advancing said cannula together with said inner needle outside distal tip of said introducer, wherein said tip of said inner needle is secured inside said cannula near the distal end of said cannula;
c) expanding said expandable device by said expanding-mechanism, whereby said expandable device exerts pressure onto portions of said mammalian tissue, wherein said mammalian tissue is initially in an undistorted position;
d) advancing said cannula together with said inner needle incrementally to a preconfigured position wherein said distal end of said cannula together with said tip of said inner needle are situated beyond said undistorted position of said mammalian tissue, said mammalian tissue being pushed by said expandable device;
e) locking said inner needle and said cannula in a secured position;
f) contracting said expandable device at a preconfigured rate, while unlocking said cannula, thereby facilitating said mammalian tissue, being elastic, to spring back powerfully towards the undistorted position of said mammalian tissue, while pushing backwards said cannula, thereby penetrating said mammalian tissue by said tip of said inner needle, being disposed beyond said undistorted position of said mammalian tissue; and
g) incrementally advancing said cannula over said inner needle to a position wherein said distal end of said cannula shields said tip of said inner needle.

34. The method as in claim 33 further comprises the step of expanding said expandable device by said expanding-mechanism, thereby anchoring said cannula in position, upon transition of said expandable device from said elastic tissue into a different tissue or cavity.

35. A method for a controlled insertion of an openable introducer into and through a mammalian tissue, the method comprising the steps of:
a) providing a device including:
i. a cannula having a double lumen;
ii. an openable introducer, including an openable sharp tip having a closed state and an opened state, wherein said closed state facilitates advancement of said openable introducer through said mammalian tissue; and wherein said open state prevents advancement of said openable introducer through said mammalian tissue;
iii. an expandable device having a contracted form size, when in a contracted state, and an expanded form size, when in an expanded state,
wherein when in expanded state, said expandable device has a front end;
wherein said expanded form size is substantially larger than said contracted form size;
wherein said expandable device is securely attached on the external circumference of said cannula, proximal to said tip of said cannula;
wherein said openable introducer facilitates the introduction of said expandable device into said mammalian tissue, said expandable device being in said contracted state; and
wherein said expandable device facilitates opening of said tip of said openable introducer when said expandable device is in said expanded state wherein said tip enters a cavity or a low dense tissue;
iv. an expanding-mechanism for expanding and contracting said expandable device;
b) advancing said cannula together with said openable introducer into and inside said mammalian tissue, said openable sharp tip being in said closed state;
c) maintaining a constant preconfigured pressure inside said expandable device; and
d) advancing said cannula together with said openable introducer until said tip of said openable introducer enters into a low density tissue or cavity; thereby facilitating rapid expanding of said expandable device by said expanding mechanism, said constant preconfigured pressure inside said expandable device being larger than the external pressure applied on said tip of said openable introducer by said low density tissue or cavity.

36. The method as in claim 35, wherein said device further includes an advancement mechanism for incrementally advancing, in a controlled manner, devices selected from the group consisting of said openable introducer, said cannula and said expandable device.
